(19) United States
(12) Patent Application Publication
(10) Pub. No.: US 2025/0149127 A1
(43) Pub. Date: May 8, 2025

(54) METHOD FOR PREDICTING A TECHNICAL APPLICATION PROPERTY OF A POLYMER

(71) Applicant: BASF SE, Ludwigshafen (DE)

(72) Inventors: Volker Settels, Ludwigshafen (DE); Philipp Eiden, Ludwigshafen (DE); Andree Lischewski, Fredenbeck (DE); Andrew David Palmer, Heidelberg (DE); Astrid Niederle, Freeport, TX (US); Jessica Eleanor Bean, Ludwigshafen (DE); Hugo Vale, Ludwigshafen (DE); Robert Matthew Lee, Ludwigshafen (DE); Miriam Mathea, Ludwigshafen (DE); Gerhard Krennrich, Frankenthal (DE); Glauco Battagliarin, Ludwigshafen (DE); Michael Bernhard Schick, Ludwigshafen (DE)

(21) Appl. No.: 18/838,348

(22) PCT Filed: Feb. 16, 2023
(86) PCT No.: PCT/EP2023/053931

§ 371 Date: Aug. 14, 2024

(30) Foreign Application Priority Data
Feb. 16, 2022 (EP) 22157126.8

(51) Int. Cl. G16C 60/00; G06F 30/27; G16C 20/70

(52) U.S. Cl. CPC G16C 60/00; G06F 30/27; G16C 20/70

(57) ABSTRACT

The invention refers to an apparatus (110) for predicting a technical application property for a polymer based on a digital representation of the polymer. A digital representation providing unit (111) provides a digital representation of the polymer indicative of polymer descriptors. The polymer descriptors are indicative of parameters quantifying physicochemical characteristics of subgroups of the polymer. A prediction model providing unit (112) provides a prediction model adapted to predict a technical application property of the polymer based on the digital representation, wherein the prediction model is a data-driven model parametrized such that it predicts based on the polymer descriptors indicated by the digital representation the technical application property associated with the polymer. A property determination unit (113) determines the technical application property based on the provided digital representation of the polymer and the prediction model. An output unit (114) provides the technical application property.

### FIELD OF THE INVENTION

The invention relates to a method, an apparatus and a computer program product for predicting a technical application property of a polymer. Further the invention refers to a training method, a training apparatus and a computer program for training a data-driven prediction model utilizable by the method, apparatus and computer program product for predicting a technical application property of a polymer.

### BACKGROUND OF THE INVENTION

Generally, the prediction of technical application properties, for instance, a heat insulating factor, a hardness, or reflectivity, of a substance, in particular, a polymeric substance, is a challenging problem with high industrial relevance. Already existing models for performing such a prediction are often very specific to the substance and/or are computationally very expensive. Thus, they cannot be easily applied, for instance, for screening a large number of possible substances for a specific technical application property. It will thus be advantageous to provide a possibility to predict technical application properties of a polymer that allows for a robust application to new polymers and is computationally less expensive.

### SUMMARY OF THE INVENTION

It is an object of the present invention to provide a method, an apparatus and a computer program product that allow for an accurate prediction of technical application properties of a polymer that is less computational expensive and can robustly be applied to new polymers. Moreover, it is further an object of the invention to provide a training method, a training apparatus and a computer program product that allow to provide a prediction model that is usable in the method, apparatus and computer program and that can be trained to provide a good prediction accuracy by utilizing less computational resources.

In a first aspect of the present invention, a computer implemented method for predicting a technical application property for a polymer based on a digital representation of the polymer is presented, wherein the method comprises the steps of a) providing a digital representation of the polymer indicative of polymer descriptors, wherein the polymer descriptors are indicative of parameters quantifying physicochemical characteristics of subgroups of the polymer, b) providing a prediction model adapted to predict a technical application property of the polymer based on the digital representation, wherein the prediction model is a data-driven model parametrized such that it is adapted to predict, based on the polymer descriptors indicated by the digital representation the technical application property associated with the polymer, c) determining the technical application property based on the provided digital representation of the polymer and the prediction model, and d) providing the technical application property.

Since the prediction model is parameterized based on polymer descriptors are indicative of parameters quantifying physicochemical characteristics of subgroups of a polymer, i.e. based on polymer descriptors derived from parameters quantifying the physicochemical characteristics of the subgroup of the polymer, the model can learn, for instance, based on training data, how a subgroup, e.g. monomer, generally behaves after polymerization. Thus, the prediction model becomes more robust for predicting the technical application properties of polymers with new and differently composed subgroup, e.g. monomers, which were not included in the training data. Moreover, deriving a descriptor directly for a complete polymer from the recipe or from respective calculations, for instance, ensemble calculations, is generally computationally expensive, and a measurement of such characteristics is time intensive and often costly. In contrast thereto calculating subgroups with only a few atoms and bonds is less computationally expensive and even experimental measurements can be performed much easier for subgroups, or already existing data sets can be used for small compounds that can form the subgroups. Moreover, utilizing the subgroups as basis even allows to determine the descriptors for different subgroups beforehand and to store respective descriptors on a database. For processing of a polymer, i.e. for processing the digital representation of the polymer, the respective descriptors of the subgroups of the polymer can then be easily and fast received from the database. Since the number of technically relevant subgroup, e.g. monomers, is limited, this allows to obtain a property of the polymer in a less computational extensive manner and to save computational resources compared with models that are not based on subgroups. Further, the use of subgroups as basis makes it possible to address homo-, co-polymers as well as polymer blends with the prediction model.

Moreover, since the polymer descriptors of subgroups of a polymer are utilized that contain physicochemical information of the polymer, e.g. quantum chemical information of the polymer like solubility in water and octanol or molar mass of the polymer, the training of a respective biodegradation model can be improved. In particular, utilizing the polymer descriptors of subgroups allows training of such models with less training data, because some of the correlation information that needs to be learned is already presented to the model by using the polymer descriptors. This, further allows to save tests and experiment necessary for providing the training data set.

Development of new chemical products that are tailored to application requirements is a predominant problem in modern chemical industries. Recently, often further requirements are also raised, for instance, related to the environmental impact or security of the chemical product along the life cycle of the chemical product. Thus, companies developing new polymers need to invest significant resources not only in testing a potential polymer with respect to an intended application property but also in self-assessing product sustainability or security and in certification. The assessment of such conditions and the testing of the potential new polymers, including laboratory spaces and equipment, becomes costly and time consuming. Thus, there is a need to early identify an technical property of a new material in the development process. The proposed method of determining a technical application property as disclosed herein enables a faster and more efficient way of developing new materials. In an early phase, even before synthesis of the polymer, the respective technical application property can be determined. This allows to determine whether the polymer is suited for market entry. This leads to a faster time to market. This also allows to reduce waste production, because the polymer does not need to be synthesized to determine the application property. The proposed method provides a digital twin of measuring the application property of a polymer.

Further, the standard measurements and tests for a respective technical application property can be time consuming, for example, include waiting times of up to several months or even years. In particular when developing new polymers for respective applications these time consuming tests can strongly limit the development process. In this context the invention allows to provide results for a new polymer instantly strongly decreasing the time after which results are available.

Moreover, due to the incredibly high number of possible, often not even fully explored polymers, potentially suitable for a specific application, today a technical product engineer, given the technical task of finding a polymer that is not only suitable for a specific application, but also fulfills respective target properties has to synthesize and test huge amounts of possible polymers, or go through huge datasets and libraries in which potential polymers are stored in order to find a respective polymer that might fit the application. Even when utilizing sophisticated design of experiment methods, still a very high number of possible polymers has to be synthesized and experimentally tested. In this context the above described method allows to assist a user, for instance, a technical product engineer, to find potentially suitable polymers automatically and much faster. In particular, by utilizing the above method the user only has to synthesize and test potentially suitable polymers for which it has been determined that it is very likely that they fulfill the respective target property. Accordingly, unnecessary synthesizing and testing of polymers can be avoided. Thus, the method allows a user to perform a technical task of finding a polymer suitable for a technical application faster and more efficient.

The method refers to a computer implemented method and thus can be performed by a general or dedicated computer adapted to perform the method, for instance, by executing a respective computer program. The method is adapted to predict a technical application property, for example, as a value for the application property, of a polymer based on a digital representation of the polymer. In particular, the technical application property can refer to any property of a polymer and/or a substance consisting at least partly of the polymer, for example, a formulation or mixture comprising the polymer, that allows to assess a technical applicability of the respective polymer as provided after its synthesis. Preferably, the technical application property comprises at least one of mechanical properties, optical properties, physicochemical properties, chemical properties and biological properties. Generally, mechanical properties can refer to any of adhesion, tensile strength, stiffness, hardness, shrinkage, elongation, split tear, tear-strength, rebound, compressibility, abrasion, spillage, morphology, haptic properties, stress at break, elongation at break, granulometry and a degree of filling. An optical property can generally comprise any of coloration, turbidity, opaqueness, lucidity, reflection, appearance, absorption, scattering, color strength, cloud point, matting degree, optical density, spectra, refractive index. Moreover, a physicochemical property can refer to any of density, viscosity, K-value, molar weight, dispersity, molar mass distribution, particle size distribution, solubility, partition coefficients, interfacial properties, surface tension, dispersibility, storage stability, odor, segregation, coagulation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, and charge density. The chemical property can comprise any of chemical resistance, reaction timing, demolding time, growing, hard/soft segment content, crystallinity, reaction temperature, reaction pressure, decomposition, thermal decomposition, photodegradation, acidity, pKₐ, pH, moisture/water content, flammability, burning rate, selfignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value. Further, the biological property can comprise any of biodegradability, biological resistance, toxicity, biotransformation, ecotoxicology, sensitization, bacterial count, enzyme activity, distribution in environment, bioaccumulation, biological exposure.

Generally, the technical application property predicted with the method is based on the specific problem that should be solved by using the method and therefore also on the respective specific training with specific training data of the utilized prediction model. Thus, for different technical application properties different prediction models can be provided or a prediction model can be trained to predict more than one technical application property. However, the prediction follows in all cases the principles of the method as defined above.

In a first step the method comprises providing a digital representation of the polymer indicative of polymer descriptors. In particular, the providing can refer to receiving the digital representation from an input of a user using, for instance, a respective input unit. Moreover, the providing can also refer to accessing a storage unit on which the digital representation is already stored. Further, the providing can also comprise receiving polymer descriptors, for instance, via a network connection from other sources and providing the received polymer descriptors as digital representation. Moreover, being indicative of or associated with polymer descriptors of a polymer is defined as allowing to access the information of the polymer descriptors. For example, the digital representation can directly comprise the polymer descriptors, for example, in form of values for respective quantities. However, the digital representation can also be a link to the respective polymer descriptors via which the polymer descriptors can be accessed, or the digital representation can refer to an identifier that is associated with the polymer descriptors and allows to utilize a respective look up storage in order to access the polymer descriptors. Moreover, the digital representation can also refer to information that allows to derive the polymer descriptors using one or more known relations. For example, a synthesis specification or a structural formula of a polymer can be utilized as digital representation allowing to derive, using known chemical and physical laws and relations, respective polymer descriptors.

Generally, throughout the following description referring a parameter or a descriptor comprises referring both to the respective quantity and also to a specific value of the quantity if not explicitly defined otherwise. For example, a parameter being a temperature always refers to the quantity being a temperature and also to a specific value of the temperature being set for the quantity. Since in most cases the explicit value of the parameter can be different for different embodiments and application cases the value is generally not mentioned. However, providing a parameter or descriptor generally means providing the quantity, e.g. the information that a value is a temperature, and also the value of the quantity or descriptor itself.

Generally, it is preferred that the digital representation comprises the polymer descriptors, wherein the polymer descriptors are indicative of parameters quantifying the physicochemical characteristics of subgroups of the polymer. In particular, the polymer descriptors can refer to physicochemical parameters of the polymer. Generally, physicochemical characteristics can refer to physical characteristics and/or chemical characteristics, in particular, parameter, of the polymer. However, the digital representation can also be provided such that it allows to derive the polymer descriptors, for instance, by providing a representation of the polymer from which the subgroups can be determined and the polymer descriptors determined based on characteristics of the determined subgroup. A subgroup refers to a part of the polymer, wherein all subgroups of a polymer together form the polymer. Generally, a subgroup can refer to a part of the polymer, wherein the subgroups are linked together successively along a chain or network to form the polymer. Preferably, the subgroups of the polymer refer to repeating units that describe a part of a polymer which when repeated produces the complete polymer chain. However, in some cases a subgroup can also refer to a single part of the polymer that is not repeated, e.g. the end-groups of the polymer. Moreover, it is preferred that the subgroups comprise parts that are repeated. For example, a subgroup of a polymer can comprise a repeating core also present in other subgroups and further additional parts that are not present in other subgroups. Preferably, the subgroups refer to at least one of polymerized monomers or oligomer fragments. More preferably, the subgroups refer to polymerized monomers. In this context, polymerized monomers refer to monomers after their polymerization also called “mer unit” or “mer”. In particular, polymerized monomers do not refer to monomers, in particular raw materials, as present in a reaction mixture before polymerization, but refer to repeating units derived from monomers that have been changed during or after the polymerization. Thus, subgroup descriptors determined for polymerized monomers are different from subgroup descriptors determined for unreacted monomers before polymerization. It has been found by the inventors that in particular the polymerized monomers allow to determine polymer descriptors from the subgroup descriptors of the polymerized monomers that allow for an accurate determination of the technical application property. Moreover, it has been found that determining the polymerized monomers for the digital representation of a polymer is in particularly computationally inexpensive and allows for the application of effective rules. In a further preferred embodiment the subgroups, for example, referring to polymerized monomers, are provided as a molecular model which is indicative of the chemical structure of the subgroup after its polymerization. Even more preferably, the molecular model of a subgroup is chosen in a way that is suited for quantum chemical computations regarding a number of atoms and their connectivity that is representative for the properties of the subgroup within the polymer. Moreover, additionally or alternatively to a molecular model of a subgroup treating the subgroup as a monomer structure also a molecular model referring to an oligomer model can be utilized that takes into account effects of neighboring molecular structures of the subgroup in the polymer.

Generally, if the digital representation of the polymer does not directly comprise the polymer descriptors, it is preferred that the polymer descriptors are determined by determining the subgroups of the polymer. For example, respective subgroups of the polymer can be determined utilizing known methods. However, it is preferred that the determination of the subgroups of the polymer is performed in accordance with later described embodiments of the invention. In particular, it is preferred that the subgroups are determined such that between atoms of different subgroups in the polymer the bond is as least polarized as possible and, preferably, with a bond order as small as possible (e.g. a CC single bond). Additionally, it is preferred that the subgroups representing a polymer comprise the same number of active non-hydrogen-atoms then the polymer. Besides the active atoms, a subgroup can also contain further atoms, which can be ignored during computing the descriptors of the subgroup. Further, it is preferred that the subgroups are determined in a way that polymers comprising parts, which were built up with different polymerization techniques, are well covered and fulfill the foresaid conditions. An example is a polyether used as ingredient for a polyurethane. Generally, a database or archive with a plurality of reactions between polymer parts can be generated and the subgroups can be derived from the respective structure of the reactions. For example, specific chemical languishes like SMILES and SMARTS can be utilized to easily derive the subgroup of a polymer. For example, a database of reaction SMARTS can be generated and then based on the polymerization of the respective polymer a corresponding reaction SMARTS can be selected. From the selected reaction SMARTS then the SMILES of monomers of the polymer are directly derivable and, for example, RDkit can be used to determine from the SMILES of the monomers the SMILES, i.e. the number and connectivity of the atoms, of the subgroups.

The determined subgroups of the polymer are associated with subgroup descriptors indicative of parameters quantifying physicochemical, i.e. physical and/or chemical, characteristics of the subgroups in the polymer. In particular, it is preferred that if the polymer descriptors are not directly provided by the digital representation, the polymer descriptors are determined by determining a respective subgroup descriptor for each of the subgroups and to determine the polymer descriptors based on the subgroup descriptors of the subgroups, for instance, by averaging. Thus, the method preferably comprises first providing or determining for the polymer the subgroups from the digital representation of the polymer, then to determine or provide the subgroup descriptors, i.e. values of the parameters quantifying the physicochemical characteristics, of the subgroups, and then to determine the polymer descriptors based on the subgroup descriptors of each polymer.

Preferably, the polymer descriptors refer to at least one of constitutional descriptors, count descriptors, list of structural fragments, fingerprints, graph invariants, 3D-descriptors and/or higher dimensional descriptors that are indicative of parameters quantifying physicochemical characteristics of subgroups of the polymer. In a preferred embodiment the polymer descriptors refer to 3D descriptors, in particular quantum chemical descriptors. In particular, it is preferred that quantum chemical descriptors are determined for embodiments utilizing polymerized monomers as subgroups. Generally, the polymer descriptors are derived from the subgroup descriptors, thus, also the subgroup descriptors can refer to the same descriptors as stated above. In the following the possible descriptors are defined in more detail. Also in these cases the defined descriptors can refer directly to the polymer descriptors or to the subgroup descriptors.

A constitutional descriptor can refer to any of a potential, average molecular weight, polydispersity, charge, spin, boiling point, melting point, enthalpy of fusion, dissociation constant, Hansen parameter, protic, polar and dispersive contributions, Abraham parameter, retention index, TPSA, receptor binding constant, Michaelis-Menten constant, Inhibitor constant, Mutagenicity, LD50, bioconcentration, toxicity, biodegradation profile and viscosity.

A count descriptor can refer to any of a sum of atomic electro negativities, a sum of atomic polarizabilities, an amount of ingredients, a ratio of amounts of ingredients, a number of atoms and non H-atoms, a number of H, B, C, N, O, P, S, Hal and heavy atoms, a number of H-donor and H-acceptor atoms, a number of bonds, non-H or multiple bonds, a number of double, triple and aromatic bonds, a number of functional groups, a ratio of functional groups, an amount of a chemical moiety, a sum of bond orders, an aromatic ratio, a number of rings or circuits, a number of unpaired electrons, a number of rotatable bonds, rotatable bond fractions, and a number of conformers.

Polymer descriptors referring to a list of structural fragment descriptors can refer to at least one of a list of molecular fractions, a list of functional groups, a list of chemical moieties, a list of bonds, and a list of atoms. Fingerprint descriptors comprise preferably, at least one of MACCS keys, preferably, in bit format or total amount format, Morgan and other circular fingerprints, preferably, in bit format or total amount format, topological torsion, atom pairs, infrared and related spectra, fingerprint count, PubChem fingerprint, substructure fingerprint, and Klekota-Roth fingerprint. Graph invariants/topological indices descriptors comprise preferably at least one of topostructural indices and topochemical indices.

In a preferred embodiment the polymer descriptors are 3D descriptors comprising at least one of a volume as sum overall atoms, a mean volume per atom, an area as sum overall atoms, an area as mean per atom, an area over all atoms, an area as mean per atom, a solvent accessible surface, a dispersion energy, a dielectric energy, a H-donor, H-acceptor, polar and non-polar surface area, an atom resolved H-donor, H-acceptor, polar and nonpolar surface area, a shape, a sphericity, dipole and higher electric moments, polarizability, dielectric energy, protic, polar and non-polar surface area, orbital energies and orbital gaps, ionization energy, electron affinity, hardness, electronegativity, electrophilicity, excitation energies and intensities, infrared and ultraviolet absorption bands, reactivity measurements, redox potential, bond criterial points, partial charges, charge surface areas, atomic orbital contributions, bond orders, atom radius. In particular, it is preferred that the polymer descriptors refer to 3D descriptors comprising at least one of a sum of a volume over all atoms, a mean of a volume per atom, a sum of the area over all atoms, a mean of an area per atom, a solvent accessible surface, a dispersion energy, a dielectric energy, a H-donor, H-acceptor, polar and/or non-polar surface area, atom resolved H-donor, H-acceptor, polar and/or non-polar surface area, shape, sphericity, cone angles, polarizability, dielectric energy, protic, polar and/or non-polar surface area, excitation energies and intensities, infrared and/or UV absorption bands, reactivity measurements, particle charges and/or charge surface areas. A preferably utilized higher dimensional descriptor can comprise at least one of a conformational partition function, solubility, vapor pressure, activity coefficient, diffusion coefficient, partition coefficient, interfacial activity, rotational constant, moment of inertia, radius of gyration, compositional drift of polymer, density, viscosity, conformer weighted volume and area, conformer weighted H-donor, H-acceptor, protic, polar and/or non-polar surface area, charge distribution, conformational dipole moment and molecular refraction. Preferably higher dimensional descriptors are utilized that comprise at least one of solubilities, vapor pressure and activity coefficients, interfacial activity, conformer weighted H-donor, H-acceptor, protic, polar and non-polar surface area, and charge distribution.

The method further comprises providing a prediction model adapted to predict a technical application property of the polymer based on the digital representation. The prediction model is a data-driven model. In particular, the term “data-driven” is used here to emphases that the model is mainly based on respective data input and not, for instance, on intuition, personal experience or knowledge. Preferably, the prediction model refers to a machine learning based model that is based on known machine learning algorithms, like neural networks, regression models, classification algorithms, etc. It has been found that for most applications, in particular, regression models based on Random forests, LASSO, Ridge Regression and MARS algorithms are suitable, whereas for classification models, in particular, random forests and SVM algorithms are suitable. In a preferred embodiment a neural network algorithm is used. In particular, the prediction model is parameterized such that it can predict based on the polymer descriptors indicated by digital representation the technical application property associated with the polymer. Generally, the prediction model can be parameterized during a training process in which polymer descriptors derived from parameters quantifying the physicochemical characteristics of subgroups of training polymers are utilized together with corresponding technical application properties of the training polymers. Based on such training data set the respective parameters of the data-driven model can be determined such that the prediction model is also able to determine technical application properties of polymers that are not part of the training data set.

Further, the method comprises determining the technical application property based on the provided digital representation of the polymer and the prediction model. In particular, if the digital representation of the polymer comprises the polymer descriptors the polymer descriptors are provided as input to the prediction model, wherein the prediction model then provides the predicted technical application property as output. If the digital representation does not directly comprise the polymer descriptors the determining of the technical application property can comprise also determining the polymer descriptors, for instance, as described above. The such determined polymer descriptors can then be provided to the prediction model as input. The predicted technical application property can then be provided, for instance, to an output unit or to a computing unit for further processing. Preferably, the providing of the technical application property leads to a further processing with the technical application property. In such a case the providing as individual step can be omitted and replaced by processing the technical application property.

Preferably, the processing of the technical application property comprises determining control signals for controlling a production process based on the determined estimated technical application property. The production process can refer to a production process of the polymer itself or can refer to a production process of a product in which the polymer is utilized. For example, if the determined technical application property refers to an ignition temperature of a polymer that should be utilized in a production process, the generation of the controlling signals can comprise generating controlling signals that ensure that the production process is operated such that the polymer is always below the determined ignition temperature. In a preferred embodiment the control signal is indicative of a machine executable synthesis specification of the polymer, in particular, when a comparison indicates that the determined technical application property of the polymer lies within a predetermined range around a provided target technical application property. Preferably, in this embodiment a further feedback loop is provided. In particular, the result of utilizing the control signals, for instance, the result of the controlled production process, can be monitored and the result of the monitoring can be compared with the expected result determined by the prediction model. Based on this comparison the prediction model can be retrained, if necessary. For example, if the control signal is indicative of a machine executable synthesis specification, the polymer produced based on the control signals can automatically be subjected to a monitoring process measuring, for instance, the respective technical application property of the polymer and optionally also further characteristics, like a polymer structure. These measurements can then be compared with the expected results predicted by the prediction model. If the comparison indicated a difference above a predetermined threshold the measurements can be directly utilized for retaining the prediction model.

Moreover, the process of processing the technical application property can also refer to a step of selecting one or more polymers based on respectively determined technical application properties. For example, if for a plurality of potential polymers respective technical application properties have been determined the selecting can comprise comparing the technical application properties of the different polymers to predetermined selection criteria and select the polymers for which the predicted technical application properties fulfill these criteria. In particular, in an embodiment the method comprises receiving a target technical application property for a polymer and comparing the received target technical application property with a predicted technical application property and providing depending on the comparison a control signal. The control signal can refer to any signal that allows for a further control of a technical system. For example, the control signal can be adapted to control an interface for providing the result of the comparison on the interface. In a preferred embodiment the comparison refers to a validation of the target technical application property, wherein the validation is positive if the predicted technical application property falls within a predetermined range around the target application property. In this case the control signal can be adapted to simply control a user interface to provide an indication of a positive or negative validation result. However, preferably, the control signal refers to a recipe, i.e. synthesis specification, of the one or more polymers which fulfill the specified target property, i.e. which are validated positively. A recipe, i.e. synthesis specification, is generally defined as an instruction on how a polymer can be synthesized. In particular, the recipe comprises the starting substances and the respective parameters for polymerization from the starting substances. Preferably, the control signals comprise a recipe in a form that directly allows an automatic controlling of respective industrial system or labor equipment for producing the polymer. In particular, it is preferred that the control signal is indicative of a machine executable synthesis specification of the polymer, when the result of the comparison refers to the determined technical application property being within a predetermined range around the target technical application property.

In a preferred embodiment the method further comprises providing as digital representation of the polymer a synthesis specification and determining the polymer descriptors from the synthesis specification. In particular, the synthesis specification, i.e. recipe, comprises information on the polymer synthesis of the polymer, for instance, on the starting substance and process by which respective starting substances are covalently bonded to form the polymer chain or network of the polymer. The method then comprises determining the polymer descriptors from the synthesis specification. In particular, from a synthesis specification the subgroups can be determined and the polymer descriptors can then be determined based on subgroup descriptors of the subgroups, for instance, from a database or utilizing known descriptor determination algorithms. In a preferred embodiment further from the synthesis specification utilized catalysts and/or none reactive process ingredients are determined. In this case it is preferred that this information is also utilized together with the polymer descriptors by the prediction model for predicting the technical application property. Preferably, descriptors are also determined for the catalysts and/or none reactive process ingredients and the respective descriptors are also used for determining the descriptors of the polymer. Preferably, the descriptor for the catalysts and/or none reactive process ingredients refers to an amount of the respective ingredient, for example, a molar mass, a molar percentage, etc. and is taken into account for determining a polymer descriptor for the polymer.

In a preferred embodiment, the method comprises i) determining from digital representation, in particular the synthesis specification of the polymer polymerized monomers of the polymer, ii) determining for the determined polymerized monomers one or more subgroup descriptors, and iii) determining the polymer descriptors based on the determined subgroup descriptors. Preferably, the determined subgroup descriptors and polymer descriptors refer to quantum chemical descriptors.

In a preferred embodiment, the determining of the polymer descriptors from the synthesis specification comprises identifying types and amounts of subgroups based on the synthesis specification, for instance, as descriptors of the subgroups, and determining the polymer descriptors based on the identified types and amounts of subgroups. Generally, the types of subgroups can refer to predetermined types or classes that are associated with specific physicochemical characteristics, i.e. descriptors, of the subgroups, and thus with specific physicochemical characteristics of a polymer comprising these subgroups. However, since the general physicochemical characteristics of a polymer and hence the polymer descriptors can also depend on the amount of a subgroup present in the polymer that amount can also be taken into account. In a preferred embodiment the determination of the type and amount of subgroups takes into account information provided by the synthesis specification indicative of the type of polymerization. The information on the type of polymerization that can be utilized can refer, for instance, to whether the polymerization refers to a polycondensation, polyaddition, radical polymerization, cationic polymerization, anionic polymerization, or coordinative chain-polymerization. Preferably, for each type of polymerization rules are predetermined that can be applied to determine the subgroups of the polymer. For example, rules can be predetermined that determined which functional groups and/or chemical moieties of monomers in the synthesis specification react with which prioritization to which functional groups and/or chemical moieties of the synthesized polymer. The rules can be based, for instance, on kinetic considerations. Based on the number and type of polymerized functional groups and/or chemical moieties the subgroups can be determined and a number and type of the subgroups can be calculated.

In an embodiment the determination of the amount of subgroups comprises determining the amount of at least one of, amide, ester, thioester, carbonate, ether, amine, urea, urethane, thiourethane, isocyanurate, biuret, allophanate, acetal, Michal-adduct, radically polymerized double bond, siloxane, silane, silazane, phosphazene groups as well as residual amine, aldehyde, ketone, epoxide, aziridine, isocyanate, alcohol, thiol, carboxylic acid, acyl halogenide, α,β-unsaturated carbonyl groups, α,β-unsaturated carboxyl and double bond groups in the polymer based on the synthesis specification.

In a further aspect, a computer implemented method for predicting a technical application property for a polymer is presented, wherein the method comprises the steps of a) providing, via a user interface, a synthesis specification of a polymer as digital representation, b) deriving the polymer descriptors from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups, c) utilizing a computer implemented method as described above to determine and provide the predicted technical application property of the polymer based on the polymer descriptors as digital representation, and d) providing, via the user interface, the predicted technical application property to a user

In particular, the determined parameters quantifying the physicochemical characteristics of the subgroup of the polymer can be regarded as subgroup descriptors, i.e. as descriptors of the subgroups. Respective examples, for the identification of the subgroup and the determination of the subgroup descriptors have already been given above. For example, the subgroups can be identified based on predefined rules from the information provided by the synthesis specification. Moreover, the subgroup descriptors can be determined, for example, by using known molecule simulation models, or by using respective databases on which the subgroup descriptors are already stored for a plurality of possible subgroups.

In a further aspect a computer implemented training method for training a data-driven based prediction model for parameterizing the prediction model is presented, wherein the training method comprises the steps of i) providing training data comprising a) polymer descriptors for each of the training polymers, wherein the polymer descriptors are indicative of parameters quantifying physicochemical characteristics of subgroups of a respective training polymer, and b) a technical application property associated with each training polymers, ii) providing a data-driven based trainable prediction model, iii) training the provided data-driven based prediction model based on the provided training data such that the trained prediction model is adapted to predict a technical application property of a polymer based on polymer descriptors, and iv) providing the trained prediction model.

In a further aspect a computer implemented optimization method for optimizing a synthesis specification of a polymer is presented, wherein the method comprises a) receiving, via an interface, i) a synthesis specification of a polymer to be optimized, ii) a target technical application property with respect to which the synthesis specifications is to be optimized and iii) one or more optimization constrains, wherein the constrains are indicative of constrains with respect to the realization of the synthesis specification, b) optimizing the synthesis specification of the polymer with respect to the target technical application property and the optimization constrains, wherein the optimization comprises I) deriving polymer descriptors of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups, II) utilizing a method as described above by providing the polymer descriptors as digital representation to determine and provide a predicted technical application property of the polymer based on the digital representation, III) comparing the predicted technical application property with the target technical application property and determining i) the synthesis specification as the optimal synthesis specification if the predicted technical application property lies within a predetermined range around the target application property, and ii) an amended synthesis specification taking the optimization constraints into account and repeating the optimization if the predicted technical application property lies outside a predetermined range around the target application property, and IV) generating a control signal based on the optimal synthesis specification.

Generally, the constrains can refer to all constrains that limit the realizable solutions. For example, the constrains can refer to constrains provided by the technical details of an industrial plant that is to produce the optimized polymer, like a limit in the possible temperature range, in a flow rate of a preproduct, in particular, a reactant, in the availability of specific substances, like catalysts, necessary for producing a specific polymer, etc. For the optimization any known iterative optimization methods can be utilized that allow for an effective search of the polymer space limited by the respective constraints, for a polymer that fulfills the technical application property. For determining an amended syntheses specification, for example, Bayesian optimizing methods can be utilized. Moreover, rules can be used for adding or removing monomers from the synthesis specification. Generally, the determining of the amended synthesis specification can refer to a purely automated process or can refer to an interactive process, in which a user interacts with the computer system, for instance, by acknowledging or marking an amended synthesis specification.

In a further aspect of the invention a method for generating a control signal, i.e. control data, suitable for manufacturing/producing a polymer is presented, wherein the method comprises a) receiving, via an interface, i) a potential target synthesis specification of a potential target polymer, ii) a target technical application property to be fulfilled by the target synthesis specification, and iii) one or more constrains, wherein the constrains are indicative of constrains with respect to the realization of the target synthesis specification, b) determining a target synthesis specification of a target polymer with respect to the target technical application property and the constrains, wherein the determination comprises I) deriving polymer descriptors of the potential target polymer from the potential target synthesis specification by i) identifying subgroups of the potential target polymer in the potential target synthesis specification, ii) determining parameters quantifying physicochemical characteristics of the subgroups of the potential target polymer, and iii) determining descriptors of the potential target polymer based on the parameters of the subgroups, II) utilizing a method as described above by providing the polymer descriptors as digital representation to determine and provide a predicted technical application property of the potential target polymer based on the digital representation, III) comparing the predicted technical application property with the target technical application property and determining i) the potential target synthesis specification as the target synthesis specification if the predicted technical application property lies within a predetermined range around the target application property, and ii) an amended potential target synthesis specification taking the constraints into account and repeating the determination if the predicted technical application property lies outside a predetermined range around the target application property, and IV) generating a control signal based on the target synthesis specification.

In a further aspect of the invention an apparatus for predicting a technical application property for a polymer based on a digital representation of the polymer is presented, wherein the apparatus comprises a) digital representation providing unit for providing a digital representation of the polymer indicative of polymer descriptors, wherein the polymer descriptors are indicative of parameters quantifying physicochemical characteristics of subgroups of the polymer, b) a prediction model providing unit for providing a prediction model adapted to predict a technical application property of the polymer based on the digital representation, wherein the prediction model is a data-driven model parametrized such that it predicts based on the polymer descriptors indicated by the digital representation the technical application property associated with the polymer, c) a property determination unit for determining the technical application property based on the provided digital representation of the polymer and the prediction model, and d) an output unit for providing the technical application property.

In a further aspect of the invention an interface system for predicting a technical application property for a polymer is presented, wherein the system comprises a) an interface adapted to receive a synthesis specification of a polymer, b) a deriving unit for deriving polymer descriptors of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups, c) a connection unit for providing the polymer descriptors as digital representation to an apparatus as described above to determine and provide the predicted technical application property of the polymer based on the digital representation, and receiving the predicted technical application property from the apparatus for providing the technical application property to a user via the interface.

In a further aspect of the invention a training apparatus for training a data-driven based prediction model for parameterizing the prediction model is presented, wherein the training apparatus comprises i) a training data providing unit for providing training data comprising a) digital representations of a plurality of training polymers comprising polymer descriptors for each of the training polymers, wherein the polymer descriptors are indicative of parameters quantifying physicochemical characteristics of subgroups of a respective training polymer, and b) a technical application property associated with each training polymers, ii) a model providing unit for providing a data-driven based trainable prediction model, iii) a training unit for training the provided data-driven based prediction model based on the provided training data such that the trained prediction model is adapted to predict a technical application property of a polymer based on a digital representation, iv) a trained model providing unit for providing the trained prediction model.

In a further aspect of the invention, an optimization system for optimizing a synthesis specification of a polymer is presented, wherein the system comprises a) an interface adapted to receive i) a synthesis specification of a polymer to be optimized, ii) a target technical application property with respect to which the synthesis specifications is to be optimized and iii) one or more optimization constrains, wherein the constrains are indicative of constrains with respect to the realization of the synthesis specification, b) an optimization unit for optimizing the synthesis specification of the polymer with respect to the target technical application property and the optimization constrains, wherein the optimization comprises I) deriving a digital representation of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups, Il) providing the digital representation to an apparatus as described above to determine and provide a predicted technical application property of the polymer based on the digital representation, III) comparing the predicted technical application property with the target technical application property and determining i) the synthesis specification as the optimal synthesis specification if the predicted technical application property lies within a predetermined range around the target application property, and ii) an amended synthesis specification taking the optimization constraints into account and repeating the optimization if the predicted technical application property lies outside a predetermined range around the target application property, and IV) a control signal generation unit for generating a control signal based on the optimal synthesis specification.

In a further aspect of the invention a computer program product for predicting a technical application property for a polymer is presented, wherein the computer program product comprises program code means for causing the apparatus, as described above, to execute the method, as described above.

In a further aspect of the invention a computer program product for training a machine learning based prediction model is presented, wherein the computer program product comprises program code means for causing the training apparatus, as described above, to execute the training method, as described above.

In a further aspect of the present invention, a computer implemented method for determining a technical application property for a polymer based on a digital representation of the polymer is presented, wherein the method comprises the steps of a) providing a digital representation of the polymer indicative or associated with physicochemical characteristics of subgroups of the polymer, b) providing a prediction model adapted to determine a technical application property of the polymer based on the digital representation, wherein the prediction model is a data-driven model parametrized such that it is adapted to determine, based on the physicochemical characteristics indicated by the digital representation the technical application property associated with the polymer, c) determining the technical application property based on the provided digital representation of the polymer and the prediction model, and d) providing the technical application property.

In a further aspect of the present invention, a system is presented, wherein the system comprises i) a control signal comprising a synthesis specification of a polymer indicating one or more ingredients for producing the polymer, wherein the control signals are generated according to the above described method, and ii) the one or more ingredients indicated by the synthesis specification in the control signal. Generally, the control signal, can be realized or refer to control data and/or a control file, for example, the control signal can be provided in a JSON format.

In a further aspect of the invention, a use of a control signal generated according to the above described method for controlling a production process, in particular, a production process comprising the production of a polymer is presented.

In a further aspect of the invention, a control signal is presented, wherein the control signal is generated according to the above described method. Preferably, the control signal comprises a machine executable synthesis specification for producing a polymer.

It shall be understood that the methods as described above, the apparatuses as described above and the computer program products as described above have similar and/or identical preferred embodiments, in particular, as defined in the dependent claims. Moreover, also the training method as described above, the training apparatus as described above, and the training computer program product as described above have similar and/or preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system comprising an apparatus for predicting a technical application property for a polymer,

FIG. 2 shows schematically and exemplarily a flow chart of a method for predicting a technical application property for a polymer,

FIG. 3 shows schematically and exemplarily a flow chart of a method for training a prediction model for predicting a technical application property of a polymer,

FIG. 4 shows schematically and exemplarily a flow chart of a detailed embodiment of a method for predicting a technical application property for a polymer,

FIGS. 5 and 6 show schematically and exemplarily a flow chart of a detailed embodiment of a method for determining a target polymer.

FIGS. 7 to 9 show schematically and exemplarily a block diagram of a system architecture of a system and apparatus for predicting a technical application property for a polymer,

FIG. 10 shows schematically and exemplarily polymerized monomers derived from a provided number of monomers for a polycondensation,

FIG. 11 shows schematically and exemplarily polymerized monomers derived from a provided number of monomers for a polyaddition,

FIG. 12 shows schematically and exemplarily polymerized monomers derived from a provided number of monomers for a vinylic polymerization,

FIG. 13 shows schematically and exemplarily polymerized monomers derived from a provided number of monomers for a block-wise polyalkoxylation,

FIG. 14 shows schematically and exemplarily polymerized monomers derived from a provided number of monomers for a poly-Michael-addition,

FIG. 15 shows schematically and exemplarily polymerized monomers for polysiloxanes, and

FIGS. 16 and 17 show exemplarily and schematically possible user interfaces.

### DETAILED DESCRIPTION OF EMBODIMENTS

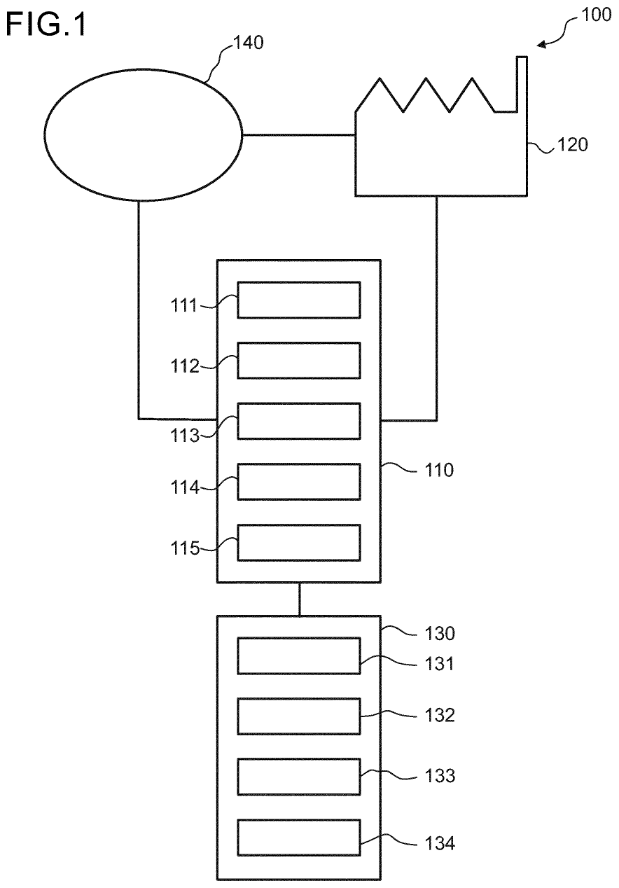

FIG. 1 shows schematically and exemplarily an embodiment of a system 100 comprising an apparatus 110 for predicting a technical application property of a polymer based on a digital representation of the polymer. Further, the system 100 comprises a training apparatus 130 for training a prediction model utilized in the apparatus 110, a database 140 on which prediction results of technical application properties for polymers can be stored, and a production system 120 for producing a product that can be controlled utilizing the predicted technical application property.

The apparatus 110 comprises a digital representation providing unit 111, a prediction model providing unit 112, a property determination unit 113 and an output unit 114. Moreover, the apparatus can optionally comprise a control unit 115 that can be adapted to provide control signals for controlling a production process of the production system 120. The digital representation providing unit 111 is adapted to provide a digital representation indicative of polymer descriptors of a polymer for which a technical application property should be predicted. The digital representation providing unit 111 can refer, for instance, to an input unit into which a user can input the respective digital representation. Moreover, the input unit can refer to or be part of a user interface that allows the user to interact with the apparatus 110 and/or the database 140. However, the digital representation providing unit 111 can also refer to or be communicatively coupled with a storage unit on which the digital representation of the polymer is already stored. Generally, the digital representation can directly comprise the polymer descriptors that are indicative of parameters quantifying physicochemical characteristics of subgroups of the respective polymer. However, instead of directly providing the polymer descriptors also a synthesis specification of the polymer can be provided. In this case, it is preferred that the digital representation providing unit 111 is further adapted to determine the polymer descriptors from the synthesis specification. In particular, it is preferred that the digital representation providing unit 111 is adapted to identify from the synthesis specification types and amounts of subgroups of the polymer and to determine the polymer descriptors based on the identified types and amounts of subgroups. In particular, the digital representation providing unit 111 can be adapted to determine for each identified subgroup respective subgroup descriptors, for instance, by accessing a database on which for a plurality of the most relevant subgroups respective descriptors are stored. The descriptors of the polymer can then be determined based on the subgroup descriptors of the subgroups and preferably, also on the determined amount and type of the subgroups, for example, by weighted averaging of the subgroup descriptors of the subgroups. More details with respect to the determination of the descriptors of the polymer are provided below in the detailed examples. The digital representation providing unit 111 is then adapted to provide the digital representation comprising the polymer descriptors, for instance, to the property determination unit 113.

The prediction model providing unit 112 is adapted to provide a prediction model adapted to predict the technical application property of the polymer based on the digital representation. Again, the prediction model providing unit 112 can comprise or refer to an input unit through which the prediction model can be received, for instance, by a user that inputs or indicates which prediction model should be used. Moreover, the prediction model providing unit 112 can refer to or be communicatively coupled with a storage unit on which the prediction model is already stored. In a preferred embodiment, the prediction model providing unit 112 further refers to a selection unit that is adapted to access, for instance, a storage unit, on which a plurality of prediction models are stored and to select a respective prediction model for providing for each application case. For example, for different polymer types, and/or for different to be predicted technical application properties different prediction models can be stored on such a storage unit and the prediction model providing unit 112 acting as a selection unit can be adapted to select, for instance, based on input of the user indicating the respective polymer and the respective to be predicted technical application property, a corresponding prediction model. Moreover, the selection unit can also be adapted to allow a user to select a respective prediction model or to support a user in the selection of a respective prediction model that is suitable for an intended application. For such user interactions, for example, the above mentioned user interface can be utilized.

The prediction model is a data-driven model parameterized such that it can predict based on the digital representation, in particular, based on the polymer descriptors indicative of the physicochemical characteristics of the subgroups the technical application property associated with the polymer. In a preferred embodiment, the data-driven model refers to a machine learning model, for instance, regression model based algorithms or a classifier model based algorithms. A regression model based algorithm can be based on any of a neural network algorithm, a LASSO algorithm, a Ridge Regression algorithm, a MARS algorithm, and a Random Forest algorithm. A classifier based model algorithm can be based on any of a Random Forest algorithm and a SVM algorithm. The inventors have found that for most applications, in particular, Random Forest and MARS based algorithms are suitable.

The prediction model can be trained, for instance, utilizing training apparatus 130. In particular, the training apparatus 130 comprises a training data providing unit 131 for providing training data for training the data-driven based prediction model. The training data comprises a) polymer descriptors of a plurality of training polymers, and b) one or more technical application properties associated with each training polymer. Preferably, in the training data the technical application property provided for each training polymer refer to the same technical application property depending on for which technical application property the prediction model should be trained. For example, the technical application property can refer to an ignition point such that the technical application property provided for each training polymer comprises the value of the ignition point for each of the training polymers. If more than one technical application property should be predicted by the prediction model then the respective more than one technical application properties are provided with respect to the training polymers in the training data. Generally, the training data can be designed to cover a predetermined application space of a to be trained prediction model. For example, the training data can be designed to cover predetermined polymer types for a predetermined application. Known methods for designing and optimizing training data for a predetermined application space can be utilized such that the application space is well covered with training data and that random outliers are avoided.

Further, the training apparatus 130 comprises a model providing unit 132 adapted to provide a data-driven based trainable prediction model, for instance, a prediction model comprising parameters that can be set during the training process for training the prediction model. For example, a trainable prediction model can already be stored on a storage unit to which the model providing unit 132 can have access for providing the same. Moreover, the training apparatus 130 comprises a training unit 133 for training the provided data-driven based prediction model based on the provided training data. In particular, the training can refer to varying the parameters of the prediction model based on the respective training data until the prediction model is adapted to predict a technical application property of a polymer based on a digital representation. Generally, any know training algorithms for training data-driven, in particular, machine learning based models can be utilized. Preferably, during the training of the prediction model also the descriptors of the polymer that have the most influence on the respective property are determined and the model is then trained based on these most influential descriptors. For determining these most influential descriptors, for example, cluster analysis or PCA analysis tools can be utilized. In particular, the descriptors can be utilized to determine the application space of the training data, wherein the application space is then defined by the descriptors of the polymer and the property data. The determination of the most influential descriptors can then be performed as a dimension reduction of the application space. Then algorithms for optimizing the training data in the application space can be applied, for instance, to cover the application space with a view training data as possible.

The training apparatus 130 then comprises a trained model providing unit 134 that is adapted to provide the trained prediction model, for instance, to a storage unit on which respectively trained prediction models for different technical application properties or different types of polymers are stored. However, the trained model providing unit 134 can also be adapted to directly provide the trained prediction model, for instance, to the prediction model providing unit 112 of apparatus 110.

In all cases, the prediction model providing unit 112 is then adapted to provide a suitable trained prediction model to the property determination unit 113. The property determination unit 113 can then utilize the prediction model and the provided digital representation for determining the technical application property. In particular, the property determination unit 113 can be adapted to utilize the polymer descriptors indicated by the digital representation as input to the prediction model that has, as already described above, been trained to then provide as output a prediction for the technical application property for which it has been trained. The output unit 114 referring, for instance, to a display, can then be adapted to output the predicted technical application property. However, the output unit 114 can additionally or alternatively be adapted to provide the predicted technical application property to a database 140 for storing the polymer in association with the predicted technical application property for future usage. In particular, the output unit 114 can be adapted, if for different polymers technical application properties have already been determined and, for instance, be stored on the storage unit, i.e. database 140, to select a respective polymer based on predetermined criteria with respect to the technical application property. The output unit 114 can then be adapted to provide and/or output the selected polymer and its technical application property. This is in particular suitable in cases in which a user searches for a polymer with a specific characteristic with respect to one or more technical application properties, from a plurality of candidate polymers.

Optionally, the apparatus 110 can comprise the control unit 115 that is adapted to provide control signals based on the predicted technical application property for controlling a production process of a production system 120. In particular, it is preferred that the control unit 115 is adapted to receive a target technical application property for a polymer and to compare the received target technical application property with the predicted technical application property and to provide the control signal depending on the comparison, preferably, to provide control signals that indicate the usage or production of the polymer for which the technical application property has been predicted. Moreover, the control signals can be indicative of a machine executable synthesis specification of the polymer for which the technical application property has been predicted, when the result of the comparison refers to the determined technical application property being within a predetermined range around the target technical application property. However, the control unit 115 can also be adapted to control the production process of another product based on the predicted technical application property, for instance, to provide control signals indicative of a machine executable synthesis specification for another product utilizing or comprising the respective polymer. For example, if a polymer utilized during a synthesis of another product is predicted as comprising an ignition point with a specific value, the controlling unit 115 can be adapted to provide control signals that ensure that during the synthesis of the product the production system 120 is always operated below the respective inflammation point.

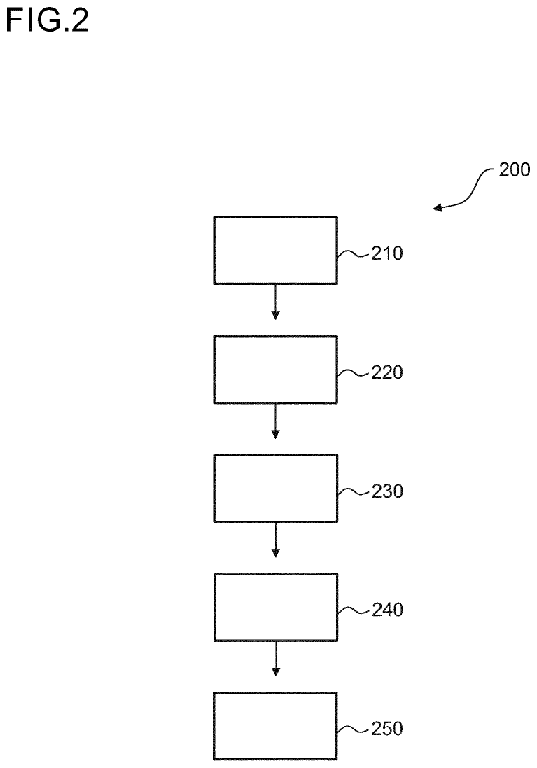

FIG. 2 shows schematically and exemplarily and a flowchart of a method for predicting a technical application property of a polymer based on a digital representation of the polymer. The method 200 comprises in a first step 210 providing a digital representation indicative of polymer descriptors of the polymer. In particular, the providing of the digital representation in this step can be in accordance with the principles described above with respect to the digital representation providing unit 111. Further, in step 220 a prediction model is provided that this adapted to predict the technical application property of the polymer based on the digital representation. As already discussed above in more detail, the prediction model is a data-driven model parameterized such that it can predict based on the polymer descriptors indicative of parameters quantifying the physicochemical characteristics of the subgroups the technical application property associated with the polymer. Generally, the step 210 and the step 220 can be perform in arbitrary order or even concurrently. In a following step 230 the technical application property is determined based on the provided digital representation of the polymer and the prediction model. The technical application property can then be provided in step 240, for instance, for outputting the technical application property on a display. Optionally, the method can further comprise in a step 250 generating control signals that allow for a controlling of a production process of a product, for instance, the polymer or a product comprising the polymer, as also already described above in more detail.

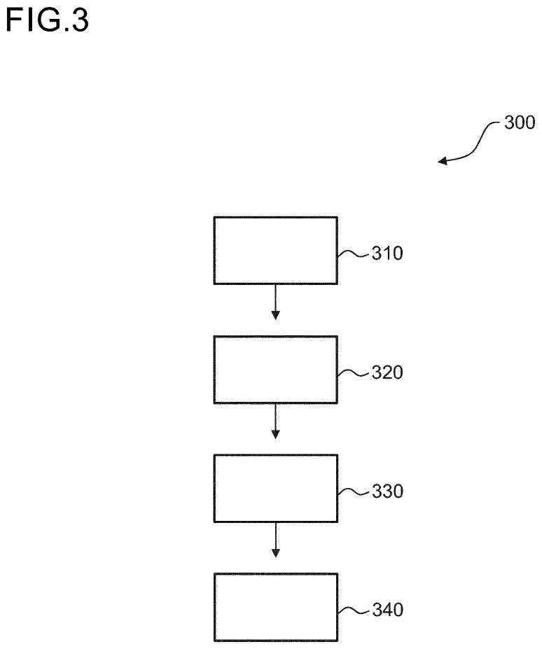

FIG. 3 shows schematically and exemplarily a flow chart of a method for training the data driven based prediction model utilized, for instance, in the method 200 discussed with respect to FIG. 2. Generally, the method 300 can be perform, for instance, by respective units of the training apparatus 130 as described with respect to FIG. 1. The method 300 comprises a step 310 of providing training data for training the data driven based prediction model. The training data comprises a) polymer descriptors of a plurality of training polymers, and b) a technical application property associated with each training polymer. In particular, the training data can be provided in accordance with the principles described above with respect to the training data providing unit 131 described with respect to FIG. 1. The method comprises further a step 320 of providing a data driven based trainable prediction model, for instance, a machine learning based prediction model like a neural network. Generally, the step 310 and the step 320 can be performed in arbitrary order or even at the same time. The method 300 then further comprises a step 330 of training the provided data driven based prediction model based on the provided training data, for instance, by varying parameters in the data driven based trainable prediction model, such that the trained prediction model is adapted to predict a technical application property of a polymer based on a digital representation of the polymer. In step 340 the trained prediction model can then be provided, for instance, by storing the trained prediction model on a storage or by directly providing the trained prediction model to the apparatus 130 as described with respect to FIG. 1.

In the following more detailed preferred examples of the above described method and the corresponding apparatus will be described in the following. In particular, the method, for example, as described above can be utilized to predict technical application properties of a polymer that belong at least to the group of a) mechanical properties, like adhesion, tensile-strength, stiffness, hardness, shrinkage, elongation, split-tear, tear-strength, rebound, compressibility, abrasion, spillage, morphology, haptic properties, stress at break, elongation at break, granulometry, degree of filling, b) optical properties, like coloration, turbidity, opaqueness, lucidity, reflection, appearance, absorbance, scattering, color strength, cloud point, matting degree, optical density, spectra, refractive index, c) physicochemical properties, like density, viscosity, K-value, molar weight, dispersity/molar mass distribution, particle size distribution, solubility, partition coefficients, interfacial properties, surface tension, dispersibility, storage stability, odor, segregation, electric conductivity, electric capacity, surface area, flow time, vapor pressure, VOC, solid content, hygroscopicity, magnetism, miscibility, thixotropy, phase transition properties, glass transition temperature, corrosion inhibition, solvent separation, aggregation, self-heating ability, impact sensitivity, loss on drying, angle of response, electrostatic charge, minimum film-forming temperature, charge density, dart drop, melt volume rate, flowability, tear propagation resistance, sealing strength, permeation, d) chemical properties, like functional group count, atom type count, functional group density, atom type density, chemical resistance, reaction timing, demolding time, growing, hard/soft segment content, crystallinity, reaction temperature, reaction pressure, decomposition, thermal decomposition, photodegradation, acidity, pKa, pH, carbon footprint, production costs, waste formation, moisture/water content, flammability, burning rate, self-ignition, flash point, formation of flammable gases, reaction to fire, deflagration rate, residual monomer count, side product formation, degree of polymerization, salt content, temperature tolerance, oxidizing properties, reduction properties, reactivity, ash content, nonvolatile matter content, stability, chelating ability, calorific value, saponification value, and e) biological properties, like biodegradability, biological resistance, toxicity, biotransformation, ecotoxicology, sensitization, bacterial count, enzyme activity, distribution in environment, bioaccumulation.

Thus, the method can be applied in a plurality of technical fields, like, agricultural polymers, coatings, dispersions, structural polymers, polymer foams, e.g. for thermal and acoustic insulation, shoe and automotive applications.

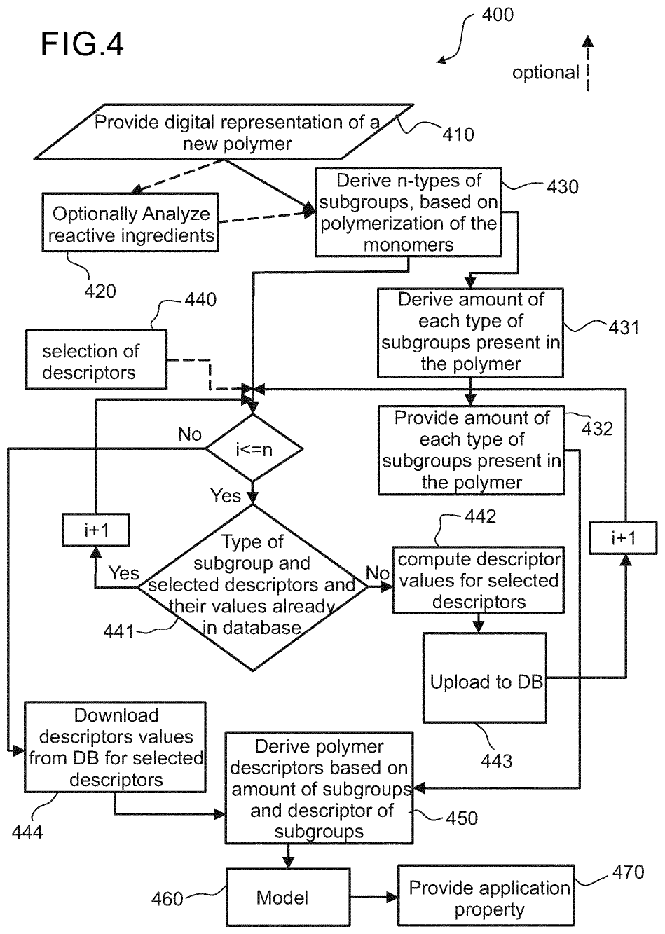

An exemplary embodiment of the method can consist out of steps described in the following. A schematic and exemplary flowchart of the exemplary embodiment of the method 400 is show in FIG. 4. In a first step 410 a digital representation of the polymer is provided. The digital representation can directly comprise the polymer descriptor, wherein in this case the steps shown in FIG. 4 until step 450 can be omitted. However, in many cases the polymer descriptors first have to be determined based on the provided digital representation referring in such a case, for example, to a recipe for the synthesis of the polymer, or to a chemical representation of the polymer indicating the chemical components and bonds in the polymer. In this step 410 the digital representation can comprise any one or more of the following information an amount of monomer components; an amount of non-monomer components, like initiators, fillers, additives; a reaction condition, like temperature, vessel, pressure, stirring rate; condition profile, e.g. temperature profile, pH value, solvents; feed profiles; type of polymerization, e.g. radical, cationic, anionic, polycondensation, polyaddition, polyether formation; post-processing, like amount of components, conditions, as well as temperature and feed profiles; type of post-processing, e.g. radical, cationic, anionic, polycondensation, polyaddition, polyether formation; chemical information on components, like mixtures, connectivity of non-polymeric pure compounds, composition of polymeric pure compounds on the basis of subgroups, connectivity of the monomers associated to the subgroups in the polymeric pure components; for bock-co-polymers also information, in which block each monomer and reactive prepolymer is incorporated; for structures/layered materials and composites also information in which phase/layer each component is included. If such information is not provided by the digital representation directly, in optional step 420, the reactive components and subgroups can also be derived from the digital representation, for example, from the recipe.

If the provided information indicates the presence of a mixture, then in a following step the mixture is decomposed into its pure components and each polymer component is treated as input polymer. Moreover, the polymer composition can also be transformed into mol %, if necessary.

In the next step 430 the polymerizable components can be transformed into subgroups, e.g. repeating units, and the subgroups are determined as different types. For example, polymerizable subgroups can be determined based on connectivity information of non-polymeric pure compounds by using SMARTS, for instance, via KNIME workflow. Also connectivity information of all possible subgroups can be derived from connectivity information of non-polymeric pure compounds by using reaction SMARTS, for example, also via KNIME workflow

After the subgroups and their types have been determined, in step 440 the type of descriptors that should be utilized can be provided. However, the descriptors can also be determined without first selecting the type of the subgroups. In order to decrease the computational resources for the method it is preferred that in a step 441 it is determined whether subgroup descriptors associated with a respective type of subgroup are already stored in a database, for example, if entries for subgroups with identical connectivity information already exist in the database. If this is the case the respective associated subgroup descriptor can be directly downloaded, for example, in step 444. If the determined type of subgroup is not stored on the database the subgroup descriptors that are associated with a respective type of subgroup can be determined, for example, in step 442. For example, either a 3D structure of respective types of subgroups can be derived based on connectivity information and an automatically computation of subgroup descriptors can be started using, for instance, a computer cluster, or already existing machine-learning predictions can be utilized as subgroup descriptors. Generally, it is preferred that if computations on new subgroups are necessary, in step 443, the results are stored in the database after the computations are finished. Optionally further subgroup descriptors can be provided from a topological analysis of the subgroups, a quantum chemical computation, a molecular dynamics computation, coarse-grained methods, finite-element computations and kinetic simulations. In particular, polymer reaction engineering methods can be used to derive subgroup descriptors that allow to take into account a microstructure of the polymer.

In step 431 the amount of subgroups, i.e. of each type of subgroup, is determined, for example based on the provided recipe information for the polymer. For example, the amount can be determined by counting an amount of polymerizable groups per polymerizable component, optionally, including prepolymers. In this case, information on polymerizable groups can be derived from non-polymeric components and the such determined amount can be added to a count of the number of, optionally, non-polymerized, polymerizable groups of the subgroups for polymeric components based on the composition of the polymeric components to determine a resulting amount. Further, it is preferred that the amount of polymerizable groups originating from agents used for post-processing after polymerization is removed from the resulting amount.

In the following some preferred exemplary rules and schemes are described for determining the subgroups and subgroup descriptors for different cases from a digital representation, for instance, a recipe of the polymer. In an embodiment in which the provided digital representation, for instance, the recipe indicates that the polymer is produced using polycondensation and polyaddition, it is preferred that the amount of reactive polymerizable groups is determined based on kinetic considerations. Preferably, all functional groups of the monomers are counted, which can react within a polycondensation or a polyaddition. These functional groups are called polymerizable groups in the following. Preferably, polymerizable groups, are categorized into two groups a nucleophilic group and an electrophilic group. All polymerizable amine, alcohol and thiol groups are then determined to belong to the nucleophilic group. All carboxylic acid, α,β-unsaturated carboxylic acid, (methyl-and ethyl-) ester, anhydride, carbonate and isocyanate groups are determined to belong to the electrophilic groups. Further, it can be assumed that amine groups react faster than alcohol groups and both faster than thiol groups during polymerization. Preferably, reactive groups are all polymerizable groups, which have polymerized in the final polymer. Preferably, non-reactive groups are all polymerizable groups, which have not polymerized in the final polymer (also called residual functional groups). Thus, if the number of polymerizable amine groups is larger than electrophilic polymerizable groups, it is preferred that the number of polymerizable amine groups is split into a non-reactive and a reactive part such that the number of reactive amine groups is equal to the number of electrophilic groups. Moreover, the amount of non-reactive alcohol groups can then be determined as being equal to the number of polymerizable alcohol groups. The amount of non-reactive thiol groups can be determined equal to the number of polymerizable thiol groups. In a case, in which the number of polymerizable amine groups is lower or equal than the number of polymerizable electrophilic groups, it is preferred to classify all polymerizable amine groups as reactive amine groups. If the number of electrophilic groups is larger than the number of polymerizable amine groups, the number of polymerizable amine groups is preferably subtracted from the number of electrophilic groups. This difference can then be regarded as the amount of remaining electrophilic groups, for this case.

If the number of polymerizable alcohol groups is larger than the remaining electrophilic polymerizable groups, the number of polymerizable alcohol groups is preferably split into a non-reactive and a reactive part such that the number of reactive alcohol groups is equal to the number of remaining electrophilic groups. The amount of non-reactive thiol groups is then equal to the number of polymerizable thiol groups. If the number of polymerizable alcohol groups is lower or equal than electrophilic polymerizable groups, all polymerizable alcohol groups can be classified as reactive alcohol groups.

If the number of remaining electrophilic groups is larger than the number of polymerizable alcohol groups, it is preferred to classify all polymerizable alcohol groups as reactive alcohol groups. The number of polymerizable alcohol groups is preferably subtracted from the number of remaining electrophilic groups. This difference then refers to the new amount of remaining electrophilic groups, for this case. If the number of polymerizable thiol groups is larger than the new remaining polymerizable electrophilic groups, the number of polymerizable thiol groups is preferably split into a non-reactive and a reactive part such that the number of reactive thiol groups is equal to the new number of remaining electrophilic groups. If the number of polymerizable thiol groups is lower or equal than electrophilic polymerizable groups, all polymerizable thiol groups can be classified as reactive alcohol groups.

If the number of remaining electrophilic groups is larger than the number of polymerizable thiol groups, the number of polymerizable thiol groups is preferably subtracted from the number of remaining electrophilic groups. This difference then refers to the amount of non-reactive electrophilic groups, for this case.

Based on the above the amount of amide, ester, thioester, urea, urethane and thiourethane groups can be determined, for example, as described below. For example, the ratio N of reacted amine groups can be determined from the amount of all reactive nucleophilic groups. The ratio O of reacted alcohol groups can be determined from the amount of all reactive nucleophilic groups. The ratio S of reacted thiol groups can be derived from the amount of all reactive nucleophilic groups. The amount of reactive carboxylic groups can then be determined as the sum of reactive carboxylic acid, α,β-unsaturated carboxylic acid, (methyl -and ethyl-) ester, anhydride and carbonate groups. The amount of reacted amide groups is then determined as equal to the amount of reactive carboxylic groups multiplied with the ratio N. Moreover, the amount of reacted ester groups is determined as equal to the amount of reactive carboxylic groups multiplied with the ratio O. The amount of reacted thioester groups is determined as equal to the amount of reactive carboxylic groups multiplied with the ratio S. Furthermore, the amount of reacted urea groups can be determined as being equal to the amount of reactive isocyanate groups multiplied with the ratio N. The amount of reacted urethane groups can be determined as being equal to the amount of reactive isocyanate groups multiplied with the ratio O, and the amount of reacted thiourethane groups can be determined as equal to the amount of reactive isocyanate groups multiplied with the ratio S.

Generally, if prepolymers are indicated by the provided recipe information, also for the prepolymers the subgroups can be determined, for example, as described above, and can be merged with the determined amount of subgroups determined for the polymer. Moreover, the amount of subgroups, which have the same connectivity and originate from the same monomer can be merged. If post-possessing agents are defined, polymerizable groups originating from post-processing agents can be merged into polymerizable groups.

In step 432 the such determined amount of subgroups can be provided and, for example, saved on the database. Before further processing the determined amount of subgroups, subgroups which are completely represented by other subgroups can be removed. Moreover, subgroups, which have the same connectivity, can be merged.

Optionally the derived amounts of subgroups can be used for a further interpretation of the polymer composition. For example, a total number of polymerized functional groups, e.g. double bonds, amine groups, alcohols groups, thiol groups, carboxylic acid groups, isocyanate groups, epoxide groups, and formed functional groups, e.g. amid groups, ester groups, thioester groups, urea groups, urethane groups, thiourethane groups, ether groups, can be determined. Also the molar weighted total number of polymerized functional groups, the mass weighted total number of polymerized functional groups, the total number of residual functional groups, e.g. double bonds, amine groups, alcohol groups, thiol, groups, carboxylic acid groups, isocyanate groups, epoxide groups, the molar weighted total number of residual functional groups, the mass weighted total number of residual functional groups, the sum of all residual functional groups, the ratio between functional groups after polymerization, the number of crosslinks in polymer, the molar fraction of crosslinks in polymer, optionally, with mass-weighting as well, the average number of atoms per subgroup, optionally, per weight as well, the average number of non-H-atoms per subgroup, optionally, per weight as well, the average number of bonds per subgroup, optionally, per weight as well, the average number of bonds between non-H-atoms per subgroup, optionally, per weight as well, the average number of rotors per subgroup, optionally, per weight as well, the average number of rotors between non-H-atoms per subgroup, optionally, per weight as well, the average number of rings per subgroup, optionally, per weight as well, the average polar surface areas per subgroup, optionally, per weight as well, the average refractivity per subgroup, optionally, per weight as well, the total number of blocks, the molar size of first block, the molar size of last block, the HLB value of polymer, optionally, with area weighted HLB value, the HLB value of block with lowest HLB value, optionally, with area weighted HLB value, the HLB value of block with largest HLB value, optionally, with area weighted HLB value, the HLB value of first block, optionally, with area weighted HLB value, the HLB value of last block, optionally, with area weighted HLB value, the mass of first block, the mass of last block, the area of block with lowest HLB value, the area of block with largest HLB value, the difference of the HLB values of the blocks, optionally, with area weighted HLB value, the hydrophilic area of the polymer, the lipophilic area of the polymer, the number of arms for ring-opening-polymerization, or the length of arms for ring-opening-polymerization can be determined.

In step 450 the determined amount and type of the subgroups and the associated subgroup descriptors can be utilized to compute the polymer descriptors. For example, the polymer descriptors can be determined by one or more of molar weighted, e.g. arithmetic, harmonic or logarithmic, averaging, mass weighted, e.g. arithmetic, harmonic or logarithmic averaging, volume weighted, e.g. arithmetic, harmonic or logarithmic, averaging, surface area weighted, e.g. arithmetic, harmonic or logarithmic, averaging of the associated descriptors of the subgroups. Moreover, the polymer descriptors can be determined by determining from the associated subgroup descriptors one or more of a molar weighted standard deviation, a mass weighted standard deviation, a volume weighted standard deviation, a surface area weighted standard deviation, a molar weighted maximum value, a mass weighted maximum value, a volume weighted maximum value, a surface area weighted maximum value, a molar weighted minimum value, a mass weighted minimum value, a volume weighted minimum value, a surface area weighted minimum value, a molar weighted sum, a mass weighted sum, a volume weighted sum, a surface area weighted sum, and a maximal difference.

In step 460 the derived or provided polymer descriptors can then be provided to the trained prediction model for predicting the application property. Generally, as already described above for predicting different polymer properties also differently trained prediction models can be utilized. However, a prediction model can also be adapted to predict more than one application property. The prediction model can be trained based on an automated statistical preprocessing of the training data, in particular, of the training polymer descriptors, for instance, utilizing feature engineering. For example, the feature engineering can comprise determining first a plurality of different polymer descriptors for the polymer, for example, based on the subgroup descriptors of the subgroups, and to preselect from this plurality of descriptors those that are with a predetermined probability relevant for a property. Based on the relevant descriptors preferably a cluster analysis is performed to identify groups of descriptors that strongly correlate. Such groups allow to select only one of the member of the groups, i.e. only one descriptor of the group, for representing the whole group of descriptors. Thus, based on the cluster analysis the number of relevant descriptors can be reduced further. Based on the remaining descriptors an application space is determined and optimized. The application of the trained prediction model is determined by the space spanned by the training data that forms the application space. This space can be optimized, for example, by amending the training data to cover the application space regularly, by removing strong outliers, by adding training data in parts of the space that are not yet covered, etc. This also allows to maximize the applicability space. The prediction model is the trained based on the optimized training data. The prediction model can generally refer to sparse, e.g. Splines, LASSO regression, PLS, and non-sparse, e.g. ridge regression, tree methods, kernel based methods, statistical learning models for relating the polymer descriptors to application properties of interest. Moreover, the prediction model can further provide a reliability estimation of the prediction depending on the respective used prediction model. In step 470 the predicted technical application property can then be provided to a user, for example, via a user interface.

The method described above can then optionally be deploy in form of a data-driven model on a polymer database, which allows to define the composition of a desired polymer in a flexible and customer-oriented way. For example, the database can be provided with a computer implemented program which allows to provide as input information, i.e. as digital representation, an amount of components, e.g. monomers and prepolymers, a type of polymerization, e.g. vinylic or polycondensation, and/or an assignment of components to certain polymer blocks, for block-co-polymers, polymer composites or polymers with compositional shift. Based on this input information the method can be adapted to derive subgroups from the user input, for instance, as described above. Further, subgroup descriptors of the subgroups can be determined, in particular, downloaded from a respective database. Based on the subgroup descriptors and the amount of subgroups the polymer descriptors can then be derived. Moreover, additional polymer descriptors can also be computed from composition information. Then the predictions of one or more polymer properties can be determined utilizing the pre-trained machine learning based prediction model based on the polymer descriptors. The property prediction can then be provided to a frontend of the polymer database application.

In a further application an embodiment of the above described method can be utilized for virtual screening using, for example, one of composition optimization, Pareto optimization, low-dimensional visualization of screened recipes, feature selection for maximal applicability domain of model, and applicability domain checker for new recipes on descriptor level.

Advantages provided by utilizing the above described method are described in the following. Many polymer properties are based on the chemical nature of the polymer. Therefore, it is advantageous to use descriptors, which reflect the chemical nature of the polymer. These are, for example, quantum chemical descriptors and topological descriptors derived from the subgroups. Frequently, polymers are made from prepolymers, and for the latter often a large variety of grades exists, each of which is only included in very few polymer samples in the historic data sets. This limits the performance of statistical models solely based on recipe information. A method based on subgroups overcomes this limitation, because the prepolymers are decomposed into subgroups as well. This helps, because the used prepolymers are often comprised of varying amounts of similar subgroups, e.g. ethylene oxide and propylene oxide based subgroups for polyalkoxylates. Consequently, the polymers in the historic data set contain similar subgroups, though the polymers contain different prepolymers. Moreover, the expression of the polymer composition in terms of subgroups allows to determine the chemical changes during polymerization. This information can be used as additional source of descriptors. These additional descriptors can in some applications be advantageous to determine application properties with the above described method. Furthermore, the above described exemplary method for deriving subgroups from other information, for instance, connectivity information of pure components, is consistent for different monomers and different types of polymerization. This allows to cover co-polymers as well as a mix of subgroups from different types of polymerization. This is advantageous, for example, if prepolymers, e.g. a polyalkoxylate, are used in a polyaddition or polycondensation.

Preferably the polymer descriptors utilized in the above described method originate from quantum chemical computations with solvation treatment. Quantum chemical computations scale very unfavorable with the system size, which makes computations on polymers or shorter monomer sequences impractical. This obstacle is solved by the above method that comprises cutting the polymer at preferably non-polarized bonds into subgroups. The resulting subgroups have a similar size than the monomers and the descriptors can be calculated using quantum chemical methods.

In particular, the above described method allows to utilize more and, in particular, more informative, i.e. more descriptive, polymer descriptors as basis for the prediction model to determine the technical application property. This allows in particular, a descriptor selection, for example, to determine polymer descriptors that are in particular suitable for prediction a technical application property. This descriptor selection provides a generalization ability already during a training of a machine learning prediction model. This results in a broader applicability domain and robustness of the prediction model. Moreover, since the prediction model is based on polymer descriptors, which are derived from subgroups, the prediction model has not to learn, how a monomer behaves after polymerization. Therefore, the prediction model is more robust for predictions for polymers including new monomers, which were not included in the training data. Moreover, predictions of polymer application properties utilizing the above described method is computationally less costly and thus can be performed generally faster than known methods. This enables polymer composition optimization, during which it is necessary to provide predictions for hundreds or thousands of different polymers.

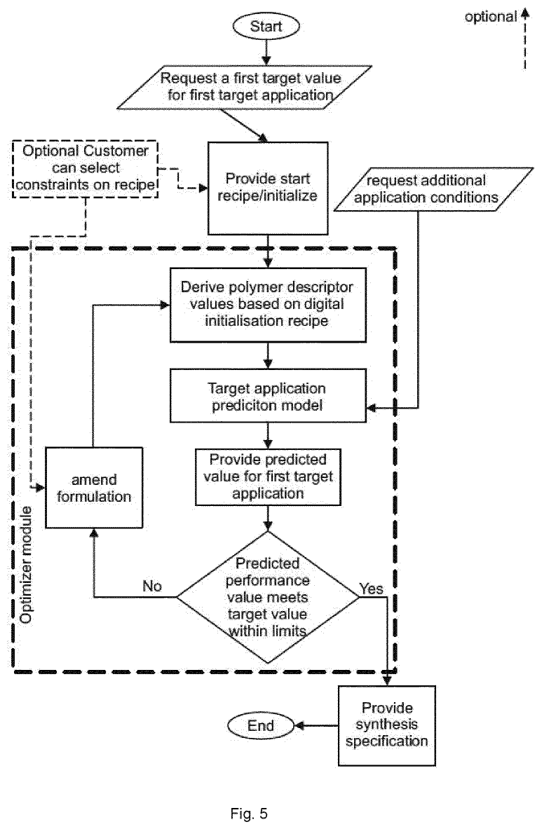

In the following, more detailed preferred examples of a preferred embodiment of the above described method and the corresponding apparatus will be described. A schematic and exemplary flow chart of an exemplary and preferred embodiment of the method is provided by FIG. 5. In this exemplary embodiment, the method starts with requesting, for instance, via a user interface, a target value for a target application. Moreover, in a next step, the optimization is initialized by providing a potential target synthesis specification, i.e. a start recipe. Optionally, constraints on the recipe, i.e. the synthesis specification, can be taken into account in this process, for instance, if a user provides such constraints. The constraints can refer, for instance, to constraints in the production of a polymer, in the starting substances that should be used for synthesizing the polymer, etc. Moreover, additional application conditions can be requested being in particular indicative of further possible information with respect to the target polymer that should be fulfilled. Based on the above steps, the optimization for determining the target polymer, i.e. the target synthesis specification, can be initialized. In a first step of the optimization, polymer descriptor values can be derived from the provided start recipe, i.e. from the provided potential target synthesis specification, for example, as described in detail with respect to FIG. 4. However, the deriving of the polymer descriptors can also refer to accessing a storage on which respective polymer descriptors for the respective potential target polymer are already stored. Moreover, if the provided digital representation of the potential target syntheses specification already comprises the polymer descriptors, this step can also be omitted. Based on the requested additional application conditions, a respective prediction model can be provided. Based on the provided prediction model and the digital representation of the potential target synthesis specification, a value for the target application for the potential target polymer can be provided. In a next step it is determined if the determined performance value, i.e. the determined technical application property, meets the target value within predetermined limits. If this is not the case, i.e. if this condition is not fulfilled, the formulation of the potential target synthesis specification is amended and a new target synthesis specification is determined optionally taking into account the constraints previously provided. The iteration can then start anew for the new potential target synthesis specification. If at one point the determined performance value meets the target value within limits, i.e. if the respective condition is fulfilled, the potential target synthesis specification is determined as the target synthesis specification and provided, for example, to a user or to a control unit for producing the respective determined target polymer.

FIG. 6 shows schematically and exemplarily a further preferred embodiment of the above method for determining a target synthesis specification with a predetermined first target technical application property, wherein in this embodiment in addition to the first target application property it is desired that the target polymer also fulfills a further second target value, i.e. target technical application property. The additional target technical application property can refer also to any technical application property. Generally, in particular, the method follows the same principles as described above with respect to FIG. 5. However, due to the additional target value, additional conditions have to be met during the optimization. Thus, in the following only the main differences with respect to the method as described above will be pointed out. In particular, in this preferred embodiment, the optimizer module does not only optimize over the first target value, i.e. over the first target application property, but also over the second target value. Preferably, also for the second target value a prediction model adapted for determining a value for the technical application property based on polymer physicochemical parameters is utilized. Thus, in addition to the method as described above for the second target application a second prediction model is provided that allows to determine an application property value based on the polymer physicochemical parameters for the second target application. The second prediction model can, for instance, be based on the same algorithm as the first prediction model, and is only trained with a different data set such that it determines another property of the polymer. The comparison then refers to not only determining whether the determined first application property meets the target application property within limits, but also whether the determined second application property value meets the target second application property value within limits. Predetermined rules can be utilized that determine for which cases the iteration is continued, i.e. a new formulation is provided as new potential target synthesis specification and for which conditions the potential target synthesis specification is determined as the target synthesis specification. For example, a user can predetermine weights for weighting to which extents which of the conditions has to be met. For instance, it can be more important for a user that the first technical application property is met, whereas the other target application property is not so important. In this case, either the limits within which the second target application property can be met can be set broader or the meeting of this condition can be weighted less strongly. If at one point of the iteration it is then determined that the conditions are met and fulfil the predetermined rules the respective potential target synthesis specification can be determined as target synthesis specification and provided as output to a user or can be utilized to generate a control file for producing the respective target polymer.

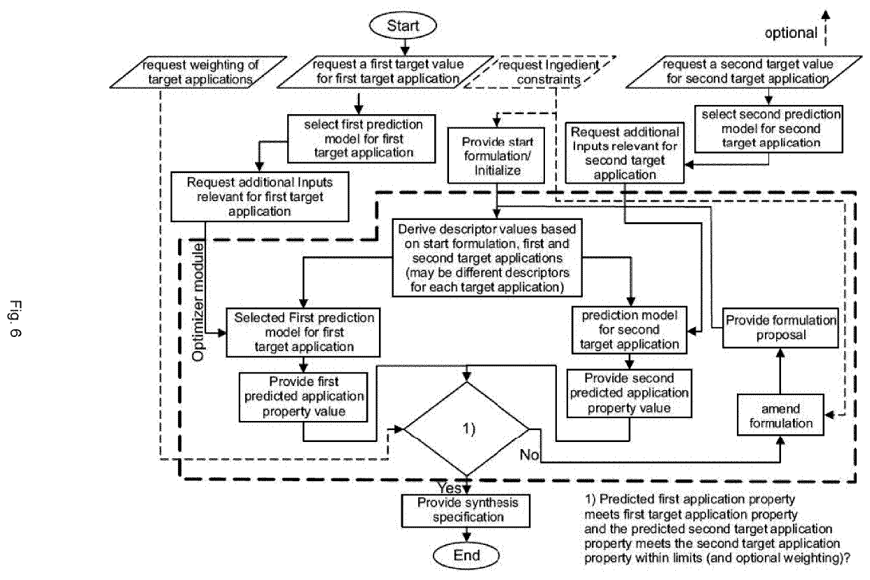

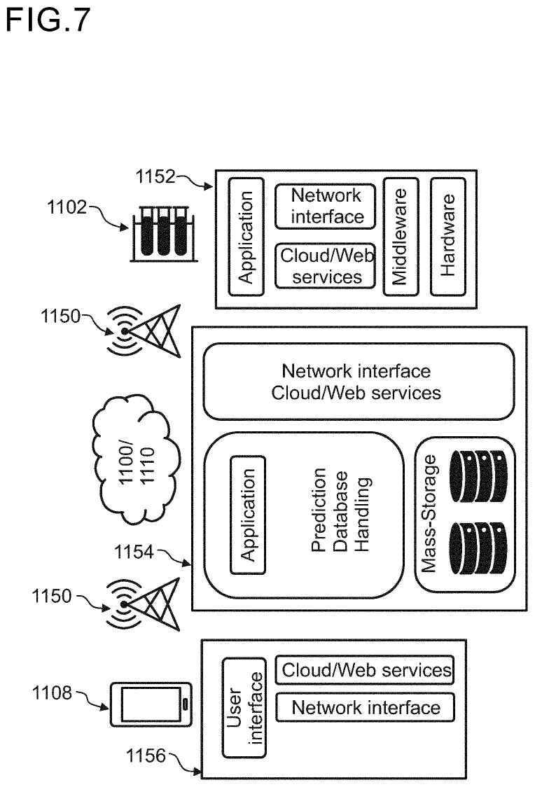

FIG. 7 illustrates a block diagram of an exemplarily system architecture of an automated laboratory system 1000 for synthesizing a polymer with a laboratory equipment control device 1102, a network 1150 and the synthesis specification, i.e. recipe, module 1100/1110, and a client device 1108. The automated laboratory system includes a laboratory equipment control device layer 1152 as part of the laboratory equipment control device 1102 as well as a synthesis specification module layer 1154 associated with the synthesis specification module and a remote control or client layer 1156 associated with the client device 1108. The laboratory equipment control device layer can be split into several hierarchical layers: the hardware, the middleware and the interface layer. The hardware layer relates to hardware resources such as sensors and actuators, in particular for controlling synthesis of a polymer. The middleware relates to any of the known middleware for laboratory or plant synthesis operations. One example is LABS/QM, providing different abstractions to hardware, network and operating system such as low-level device control and message passing. The communication layer relates to communication protocols one the protocol may be REST, which may be implemented over different transport protocols (i.e. UDP, TCP, Telemetry) that allow the exchange of messages between the laboratory equipment control device and laboratory equipment devices. Such software architecture allows to control and monitor laboratory equipment without having to interact with the hardware.

The synthesis specification module layer 1154 may include: a mass storage layer, the computing layer, the interface layer. The storage layer is configured to provide mass storage for the data-driven prediction model for providing a recipe, i.e. synthesis specification, of a polymer based on a technical application property, as described in detail above. In particular, the functions performed by the apparatus, as described above, can be provided as program code means stored on the mass storage. Furthermore, synthesis specifications for a plurality of polymers can be stored in the mass storage. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes based on target properties. Such functionalities can include determining based on a target technical application property and the prediction model a digital representation of a target polymer, generating a synthesis specification from the digital representation of the target polymer, and providing the synthesis specification as control data, i.e. control signal, to the laboratory equipment control device.

The interface layer may implement web services, network interfaces as UDP or TCP or Websocket interfaces. For communication with the laboratory equipment control device a REST API is implemented.

The client layer 1156 provides interfaces for end-users. For end-users, the client layer 1156 can run client side Web applications, which provide interfaces to the synthesis specification module layer 1154 or the laboratory equipment control device layer 1152. Users may be provided with a UI for selecting a target technical application property and a target value for this property, the target value may also comprise a range of the technical application property. In other examples, the users may be provided with a UI for selecting more than one technical application property and respective values. The applications may be configured for users to monitor and control the laboratory equipment control device and the operation remotely. In other examples, the client device layer and the synthesis specification module layer may be integrated into one device. The alternatives described here are only for illustration purposes and should not be considered limiting.

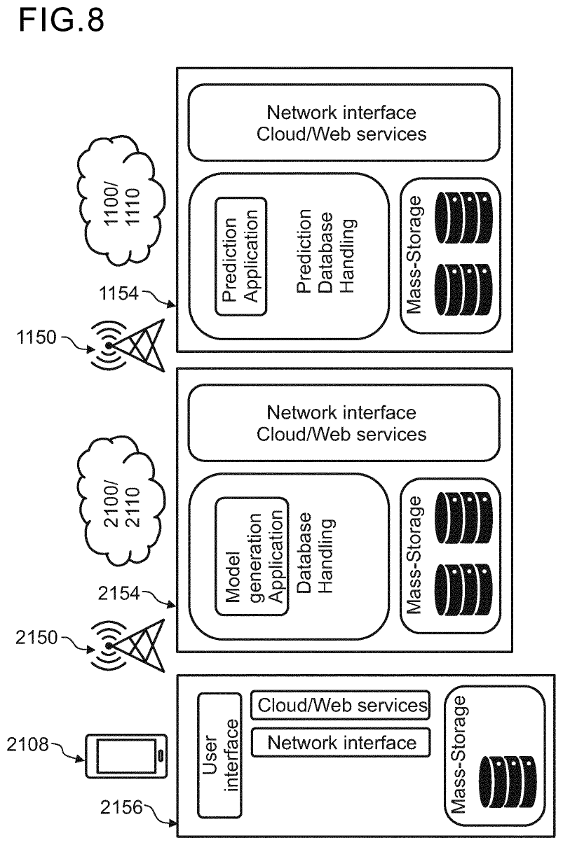

FIG. 8 illustrates a block diagram of an exemplarily system architecture of a system and apparatus for generating a prediction model for predicting a technical application property, a network 2150 and a model generating module 2100/2110 that can be regarded as or comprising a training model apparatus, a synthesis specification module 1100/1110, and a client device 2108. The system for generating a prediction model includes a model generating module layer 2154 as part of model generating module and a client layer 2156 associated with the client devices 2108.

The model generating module layer 2154 may include: a mass storage layer, a computing layer, an interface layer. The storage layer is configured to provide mass storage for the data-driven prediction model as described above. Furthermore, the mass storage is configured for storing synthesis specifications for polymers and measured technical application properties. Such data may be stored in structured databases such as SQL databases or in a distributed file system such as HDFS, NoSQL databases such as HBase, MongoDB. The computing layer may include an application layer that allows to customize the functionalities provided by standard cloud services to perform computing processes for generating a prediction model for predicting properties of polymers. Such functionalities may include receiving for at least two previously measured polymers their respective digital representation associated with a synthesis specification measurement data of at least one technical application property for each of the at least two previously measured polymers, receiving at the model generating module the digital representation of at least one unmeasured polymer, training the model according to the above described training principles based on the digital representation of the at least two previously measured polymers, the measurement data of the at least one technical application property for each of the at least two previously measured polymers, and, preferably, a similarity measure between the digital representation associated with the synthesis specification of each of the at least two previously measured polymers and the respective digital representation associated with a synthesis specification of the at least one unmeasured polymer, and providing via an output interface the prediction model for the technical application property. The model generating module layer may be configured for deploying the generated model and the synthesis specification database to the synthesis specification module layer. This may include storing the generated model and the synthesis specification database in the mass storage devices associated with the synthesis specification module.

The model generating module layer may further be configured for determining a digital representation of the polymer associated with the synthesis specification from the synthesis specification. The digital representation may include a set of polymer descriptors and polymer descriptor values associated with a synthesis specification of each measured polymer. One way of deriving these polymer descriptors can be to apply the SMILES algorithm or any other already above described principle. In case, where the model is generated based on the digital representation derived from the recipe, a relation between the synthesis specification and the descriptors may be stored in the mass storage devices associated with the model generating module. In such cases, deploying the model comprises providing that relation.

The interface layer may implement web services, network interfaces as UDP or TCP or Websocket interfaces. For communication with the client device a REST API is implemented in this example. The client layer 2156 provides access to mass storage devices, that contain synthesis specifications for polymers, and for at least two polymers at least one technical application property. The client layer further provides an interface for end-users. For end-users, the client layer 2156 may run client side Web applications, which provide interfaces to the model generation module layer 2154 or the mass storage devices associated with the client layer. Users may be provided with a UI for selecting a technical application property. The user may further be provided with a UI for selection of the synthesis specification data and the technical application property data associated with the synthesis specification data. The user interface may also provide an option for uploading the selected data to the model generating module layer and optionally an option to initiate model generation.

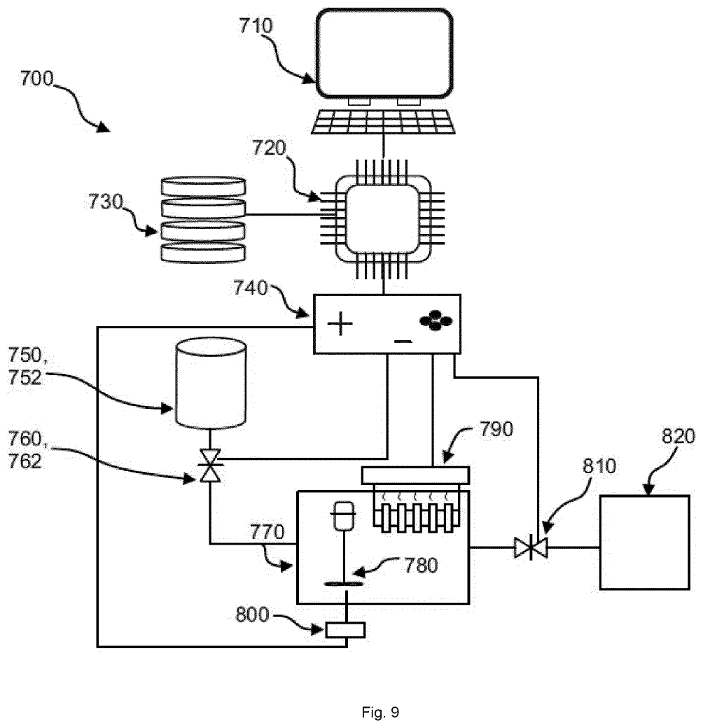

FIG. 9 shows an exemplary system 700 for producing a chemical product based on a synthesis specification generated according to the invention. In this example the system comprises a user interface 710 and a processor 720, associated with a control unit 740. The user interface 710 and the processor 720 can be associated with or realized in accordance with the principles described above, in particular, can be adapted to perform a computer implemented method to determine a target polymer and/or synthesis specification based on a predicted technical application property, as described above. The control unit 740 is, for example, configured for receiving control data, i.e. a control signal, generated according to the invention as described above, in particular, to receiving control data generated based on a synthesis specification of a polymer comprising a target technical application property. In this example the control data is provided from a data base 730, in other examples, however the control data can also be provided from a server or any other computational unit for distributing data. Vessels 750, 752 each contain a component of the chemical product, for example, pre-polymers, catalysts, etc. In general, more than two vessels are present, however, in this example for illustrative purposes only two are shown. Valves 760, 762 are associated with vessels 750, 752. Valves 750 and 752 can be controlled to dose appropriate amounts of each component into reactor 770, according to the synthesis specification. A motor 800 of a mixer 780 may also be controlled by the control unit according to the synthesis specification. An optional heater 790 may also be controlled according to the synthesis specification. Finally, an exit valve 810 in fluid communication with the reactor may be controlled by the control unit to provide the chemical product to a container or test system 820.

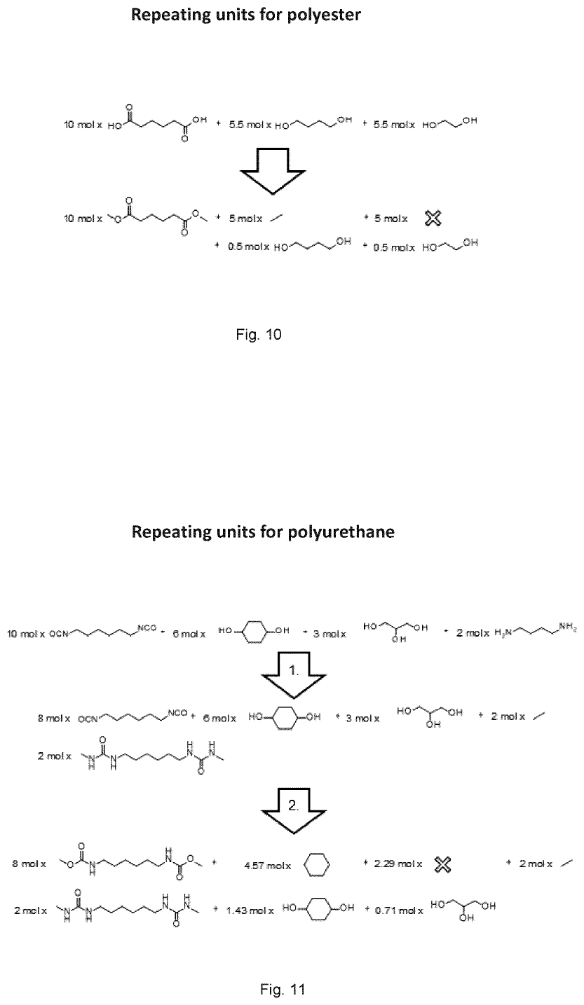

In the following some examples of subgroups determined for specific polymers are described in more detail with respect to FIGS. 10 to 15. In particular, these below provided examples on the deriving of subgroups are only exemplarily and utilize models and assumptions that lead to suitably accurate prediction results. However, also other models and assumptions can be used for deriving subgroups. In particular, utilizing kinetic models for deriving the subgroups allows for a further increase of an accuracy when determining the subgroups and also can increase the accuracy of the prediction result. FIG. 10 shows the polymerized monomers derived, for example, from a provided number of monomers for a polycondensation. In this example 10 mol adipic acid is polymerized with 5.5 mol butane-1,4-diol and 5.5 mol ethylene glycol. The 10 mol adipic acid monomers polymerize to form 10 mol adipic acid dimethyl ester, i.e. the polymerized monomers of adipic acid in the resulting polymer. The additional carbon atoms of the polymerized monomer are taken from the monomers containing alcohol groups. Accordingly, the polymerized monomer of the monomer butane-1,4-diol within the polymer chain is ethane. In the case of ethylene glycol, the polymerized monomer within the polymer chain is completely represented by the polymerized adipic acid monomer (=adipic acid dimethyl ester), which is symbolized by the cross in the scheme. This polymerized monomer of ethylene glycol can be neglected for the computation of descriptors. According to the provided number of monomers, there is an excess of 2 mol of alcohol groups compared to the acid groups. Assuming a hypothetic complete polymerization of adipic acid and an equal reactivity of the two diols butane-1,4-diol and ethylene glycol, 0.5 mol of butane diol and 0.5 mol of ethylene glycol remain unreacted. These unreacted monomers resemble polymerized monomers of the polymer chain ends. Besides this assumption it is also possible to get a more realistic distribution of polymerized monomers, for instance, with kinetic models.

FIG. 11 shows the polymerized monomers derived, for example, from a provided number of monomers for a polyaddition. In this example 10 mol hexamethylene diisocyanate is polymerized with 6 mol cyclohexane-1,4-diol, 3 mol glycerol and 2 mol butane1,4-diamine. In this example, it is assumed that the amines react preferably with isocyanates than alcohols. In a first step, 2 mol hexamethylene diisocyanate polymerize to a urea group containing polymerized monomer. Within this polymerized monomer the formed urea groups are N-substituted by methyl groups, which are taken from the amine containing compounds. Consequently, the monomer butane1,4-diamine polymerizes to ethane as polymerized monomer, since the original amine groups and two of the four carbon atoms of the monomer 1,4-butanediamine are attributed to the polymerized monomer of hexamethylene diisocyanate already. In a second step, the alcohol groups polymerize with the remaining 8 mol hexamethylene diisocyanate. There is an excess of alcohol groups compared to the number of isocyanate groups. Therefore, the 8 mol hexamethylene diisocyanate polymerize to 8 mol urethane group containing polymerized monomers which are O-substituted by methyl groups. Under the assumption of a hypothetical equal reactivity of cyclohexane-1,4-diol and glycerol, 4.57 mol polymerized cyclohexane-1,4-diol is formed, which is represented by cyclohexane. In this special case, no carbon atom is removed from the monomer, because such a removal would change the size of the ring of cyclohexane-1,4-diol. We assume a similar reactivity of all three alcohol groups of glycerol, which results in a hypothetic reaction of all three alcohol groups with isocyanates. For each reacted alcohol group, a methoxy group is removed from glycerol. Consequently, the 2.29 mol polymerized glycerol is completely represented by the urethane containing polymerized monomer of hexamethylene diisocyanate and can be neglected for the computation of descriptors. Because of the excess of alcohol groups, 1.43 mol cyclohexane-1,4-diol and 0.71 mol glycerol remain unreacted. These unreacted monomers resemble polymerized monomers of the polymer chain ends. Besides these assumptions it is possible to get a more realistic distribution of polymerized monomers with kinetic models. In this example, 2.29 mol of polymerized glycerol is formed as described above. This polymerized monomer has three reacted functional groups and acts as cross-link in the final polymer. This information on cross-links can be used as descriptor to distinguish between linear and crosslinked polymers.

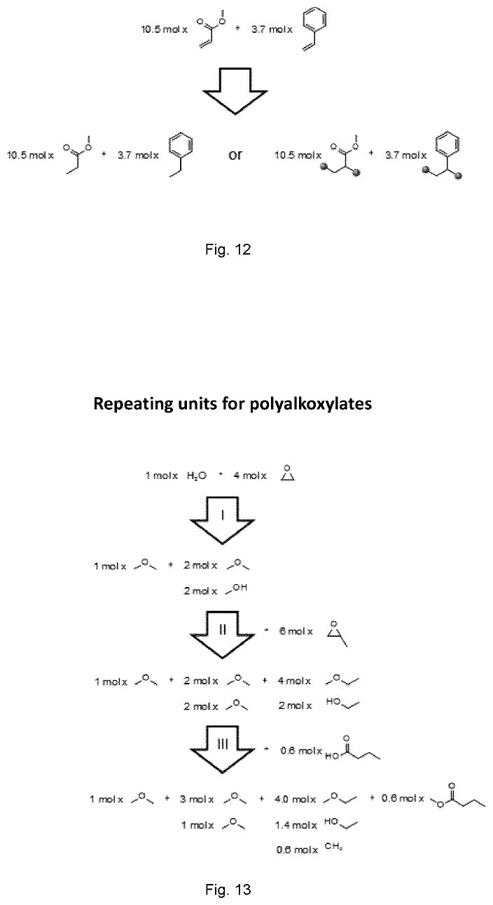

FIG. 12 shows the polymerized monomers derived from a provided number of monomers for a vinylic polymerization. In this example 10.5 mol methyl acrylate is polymerized with 3.7 mol styrene. In this example, a complete conversion of the monomers is assumed during polymerization. Therefore, 10.5 polymerized methyl acrylate and 3.7 polymerized styrene is formed. Polymerized monomers can be defined in different ways. On the left-hand side, the polymerized monomers are represented by molecular structures in which the reactive double bond of the corresponding monomers has been saturated upon a hypothetical hydrogenation (addition of 2 hydrogen atoms). For example, the monomer styrene can be represented by ethylbenzene as polymerized monomer. On the right-hand side, additional groups, e.g., methyl groups, are added, which resemble the electronic effect of the polymer chain on the polymerized monomer. However, these additional groups are preferably neglected by the descriptor computation, e.g., by ignoring their contribution to the molecular surface area. Besides this assumption it is also possible to get a more realistic distribution of polymerized monomers with kinetic models.

FIG. 13 shows the polymerized monomers derived from a provided number of monomers for a block-wise polyalkoxylation. In the first step, water is used as a model initiator representing e.g., hydroxy salts, of a polyalkoxylation in which 4 mol of ethylene oxide are polymerized. The polymerized monomer of water is dimethyl ether. After polymerization of the 4 mol ethylene oxide, two of them are located within the polymer chain and two are at the chain ends. The polymerized monomer of ethylene oxide within the chain is dimethyl ether as well. The polymerized monomer of ethylene oxide at the chain end is methanol. All polymerized monomers are attributed to the inner block of the final block-co-polymer. In a second step, 6 mol of propylene oxide reacts with the polymer from step 1. Now, the polymerized monomers at the chain end from step 1 react with propylene oxide. Consequently, these polymerized monomers at the chain end of step 1 are now located within the polymer chain and the resulting polymerized monomer is again dimethyl ether. Regarding the 6 mol of propylene oxide, 4 mol of them form polymerized monomers within the polymer chain (methoxyethane) and 2 mol form polymerized monomers at the chain end (ethanol). All polymerized monomers deriving from the monomer propylene oxide are attributed to the outer blocks of the block-co-polymer resulting after step 2. In a third step, a chain-end modification is performed of the block-co-polymer formed in the first two steps. This chain-end modification is done via a partial esterification with 0.6 mol of butyric acid, a condensation reaction under the loss of one water molecule per newly formed ester bond. The polymerized monomer of butyric acid after esterification is methyl butyrate. The additional oxygen and carbon atoms of this polymerized monomer are taken from the polymerized monomers containing alcohol groups, which are the polymerized monomers of propylene oxide at the chain end of the polymer resulting after step 2, i.e. ethanol. Accordingly, those of the polymerized monomers (ethanol) which have formed esters upon partial esterification are transformed to methane now. Besides this assumption it is possible to get a more realistic distribution of polymerized monomers also with kinetic models.

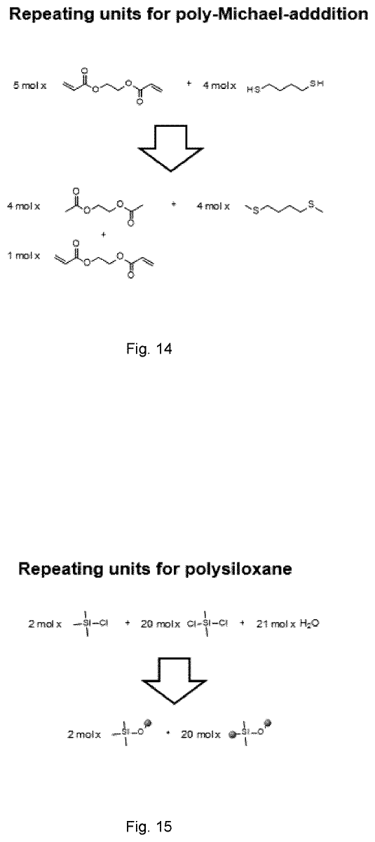

FIG. 14 shows the polymerized monomers derived from a provided number of monomers for a poly-Michael addition. In this example 5 mol ethylene glycol diacrylate is polymerized with 4 mol butane-1,4-dithiol. There is an excess of Michael-acceptor groups (acrylate groups) over Michael-donor groups (thiol groups). In this example, it is assumed that the 4 mol butane-1,4-dithiol are completely converted during polymerization. The resulting polymerized monomer of the monomer butane-1,4-dithiol is 1,4-bis(methylsulfanyl)butane. The additional carbon atoms of this polymerized monomer are taken from the monomers containing Michael-acceptor groups. Consequently, the reacted 4 mol of the monomer ethylene glycol diacrylate form 4 mol of ethylene glycol diacetate as polymerized monomers (loss of carbon atoms). The remaining excess of 1 mol ethylene glycol diacrylate monomers does not react. These unreacted monomers resemble polymerized monomers of the polymer chain ends. Besides this assumption it is also possible to get a more realistic distribution of polymerized monomers with kinetic models.

FIG. 15 shows the polymerized monomers for polysiloxanes. In this example 20 mol dichlorodimethylsilane polymerizes with 2 mol chlorotrimethylsilane and 21 mol water (hydrolysis and subsequent polyaddition). In this example, a complete conversion of the monomers is assumed during polymerization. Therefore, in this example, 20 polymerized monomers dichlorodimethylsilane within the polymer chain as well as 2 mol polymerized monomers chlorotrimethylsilane at the chain ends (represented by hydroxytrimethylsilane) are formed. Besides this assumption it is possible to get a more realistic distribution of polymerized monomers with kinetic models. In this example, it is not possible to define the polymerized monomers within the polymer chain in a way that the polymer is cut at non-polarized and homogeneous chemical bonds. Therefore, additional groups (e.g., methyl and methoxy groups) are added, which resemble the electronic effect of the polymer chain on the polymerized monomer. However, these additional groups must be neglected by the descriptor computation (e.g., by ignoring their contribution to the molecular surface area). By using methyl and methoxy groups as a model for the polymer chain, the polymerized monomer of dichlorodimethylsilane is dimethoxydimethylsilane. Alternatively, small oligomers can be used as polymerized monomer of dichlorodimethylsilane and chlorotrimethylsilane.

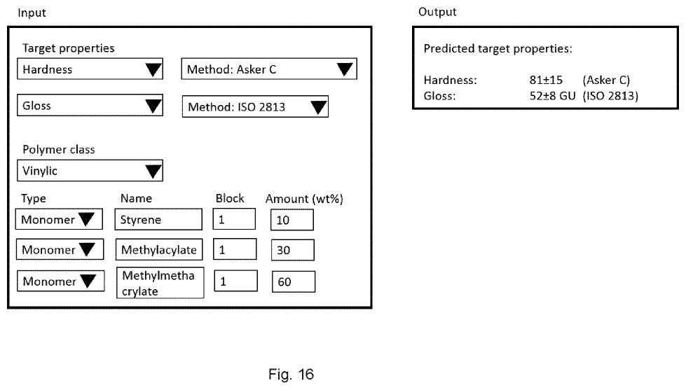

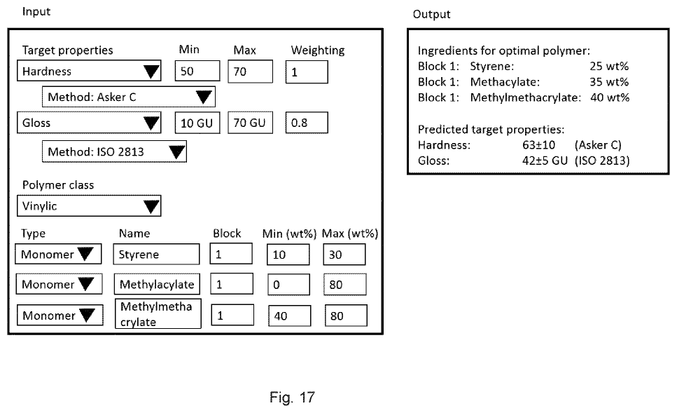

FIGS. 16 and 17 show exemplarily and schematically possible user interfaces for interfacing, for example, with a processor performing one of the above described methods for determining an application property or a target, i.e. optimized, polymer. FIG. 16 shows an exemplarily user interface for determining an application property of a polymer. In this example, an input screen is shown on the left. The input screen allows for a definition of which technical application property should be determined and optionally according to which method the application method should be measured. In this example, a hardness and a gloss should be determined. Further, the input screen can allow to provide a digital representation of a polymer for which the application properties should be determined. In this case the digital representation is defined by a polymer class being vinylic and further details referring to types of monomers and associated block and amounts for the polymer. Based on this input the target properties are then predicted in accordance with one of the methods described above. An exemplary output screen is shown on the right of FIG. 16. In this example, the output screen shows the results of the prediction of the two application properties.

FIG. 17 shows an exemplarily user interface for determining an optimized, i.e. target polymer, with a respective target technical application property. In this example, an input screen is shown on the left. The input screen allows for a definition of one or more target application properties. In this example, respective, values ranges for a hardness and a gloss of the polymer are provided. Further, the input screen allows to provide respective constrains for the target polymer. In this example, the target polymer should be constraint to a vinylic polymer class and also constraints for the monomers are defined in form of minimal and maximal values. Based on these input parameters a respective target polymer fulfilling these parameters is determined in accordance with one of the above described methods. An exemplary output screen is shown on the right of FIG. 17. In this example, the output screen provides the details of the determined target polymer together with the respective determined values for the application properties. Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the polymer descriptors and the prediction model, the determining of the technical application property, the providing the technical application property, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for predicting a technical application property for a polymer based on a digital representation of the polymer. A digital representation providing unit provides a digital representation of the polymer indicative of polymer descriptors. The polymer descriptors are indicative of parameters quantifying physicochemical characteristics of subgroups of the polymer. A prediction model providing unit provides a prediction model adapted to predict a technical application property of the polymer based on the digital representation, wherein the prediction model is a data-driven model parametrized such that it predicts based on the polymer descriptors indicated by the digital representation the technical application property associated with the polymer. A property determination unit determines the technical application property based on the provided digital representation of the polymer and the prediction model. An output unit provides the technical application property.

1. A computer implemented method for predicting a technical application property for a polymer based on a digital representation of the polymer, wherein the method comprises:
providing a digital representation of the polymer indicative of polymer descriptors, wherein the polymer descriptors are indicative of parameters quantifying physicochemical characteristics of subgroups of the polymer,
providing a prediction model adapted to predict a technical application property of the polymer based on the digital representation, wherein the prediction model is a data-driven model parametrized such that it its adapted to predict based on the polymer descriptors indicated by the digital representation the technical application property associated with the polymer,
determining the technical application property based on the provided digital representation of the polymer and the prediction model, and
providing the technical application property.

2. The method according to claim 1, wherein the method further comprises providing as digital representation of the polymer a synthesis specification of the polymer and determining the polymer descriptors from the synthesis specification.

3. The method according to claim 2, wherein the determining of the polymer descriptors from the synthesis specification comprises identifying types and amounts of subgroups based on the synthesis specification, and determining the polymer descriptors based on the identified types and amounts of subgroups.

4. The method according to claim 3, wherein the determination of the type and amount of a subgroup takes into account information provided by the synthesis specification indicative of the type of polymerization.

5. The method according to claim 1, wherein the method further comprises receiving a target technical application property for a polymer and comparing the received target technical application property with a predicted technical application property and providing depending on the comparison a control signal.

6. The method according to claim 5, wherein the control signal is indicative of a machine executable synthesis specification of the polymer, when the result of the comparison refers to the determined technical application property being within a predetermined range around the target technical application property.

7. A computer implemented method for predicting a technical application property for a polymer, wherein the method comprises:
providing, via a user interface, a synthesis specification of a polymer as digital representation,
deriving the polymer descriptors from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups,
utilizing a computer implemented method according to claim 1 to determine and provide the predicted technical application property of the polymer based on the polymer descriptors as digital representation, and
providing, via the user interface, the predicted technical application property to a user.

8. A computer implemented training method for training a data-driven based prediction model for parameterizing the prediction model, wherein the training method comprises the steps of:
providing training data comprising a) polymer descriptors for each of the training polymers, wherein the polymer descriptors are indicative of parameters quantifying physicochemical characteristics of subgroups of a respective training polymer, and b) a technical application property associated with each training polymer,
providing a data-driven based trainable prediction model,
training the provided data-driven based prediction model based on the provided training data such that the trained prediction model is adapted to predict a technical application property of a polymer based on polymer descriptors, and
providing the trained prediction model.

9. A computer implemented optimization method for optimizing a synthesis specification of a polymer, wherein the method comprises:
receiving, via an interface, i) a synthesis specification of a polymer to be optimized, ii) a target technical application property with respect to which the synthesis specifications is to be optimized and iii) one or more optimization constrains, wherein the constrains are indicative of constrains with respect to the realization of the synthesis specification,
optimizing the synthesis specification of the polymer with respect to the target technical application property and the optimization constrains, wherein the optimization comprises:
deriving polymer descriptors of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups,
utilizing a method according to claim 1 by providing the polymer descriptors as digital representation to determine and provide a predicted technical application property of the polymer based on the digital representation,
comparing the predicted technical application property with the target technical application property and determining i) the synthesis specification as the optimal synthesis specification if the predicted technical application property lies within a predetermined range around the target application property, and ii) an amended synthesis specification taking the optimization constraints into account and repeating the optimization if the predicted technical application property lies outside a predetermined range around the target application property, and
generating a control signal based on the optimal synthesis specification.

10. An apparatus for predicting a technical application property for a polymer based on a digital representation of the polymer, wherein the apparatus comprises:
a digital representation providing unit for providing a digital representation of the polymer indicative of polymer descriptors, wherein the polymer descriptors are indicative of parameters quantifying physicochemical characteristics of subgroups of the polymer,
a prediction model providing unit for providing a prediction model adapted to predict a technical application property of the polymer based on the digital representation, wherein the prediction model is a data-driven model parametrized such that it predicts based on the polymer descriptors indicated by the digital representation the technical application property associated with the polymer,
a property determination unit for determining the technical application property based on the provided digital representation of the polymer and the prediction model, and
an output unit for providing the technical application property.

11. A interface system for predicting a technical application property for a polymer, wherein the system comprises:
an interface adapted to receive a synthesis specification of a polymer,
a deriving unit for deriving a polymer descriptors of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups,
a connection unit for providing the polymer descriptors as digital representation to an apparatus according to claim 10 to determine and provide the predicted technical application property of the polymer based on the digital representation, and receiving the predicted technical application property from the apparatus for providing the technical application property to a user via the interface.

12. A training apparatus for training a data-driven based prediction model for parameterizing the prediction model, wherein the training apparatus comprises:
a training data providing unit for providing training data comprising a) digital representations of a plurality of training polymers comprising polymer descriptors for each of the training polymers, wherein the polymer descriptors are indicative of parameters quantifying physicochemical characteristics of subgroups of a respective training polymer, and b) a technical application property associated with each training polymers,
a model providing unit for providing a data-driven based trainable prediction model,
a training unit for training the provided data-driven based prediction model based on the provided training data such that the trained prediction model is adapted to predict a technical application property of a polymer based on a digital representation,
a trained model providing unit for providing the trained prediction model.

13. An optimization system for optimizing a synthesis specification of a polymer, wherein the system comprises:
an interface adapted to receive i) a synthesis specification of a polymer to be optimized, ii) a target technical application property with respect to which the synthesis specifications is to be optimized and iii) one or more optimization constrains, wherein the constrains are indicative of constrains with respect to the realization of the synthesis specification,
an optimization unit for optimizing the synthesis specification of the polymer with respect to the target technical application property and the optimization constrains, wherein the optimization comprises:
deriving a digital representation of the polymer from the synthesis specification by i) identifying subgroups of the polymer in the synthesis specification, ii) determining parameters quantifying physicochemical characteristics of the subgroups of the polymer, and iii) determining descriptors of the polymer based on the parameters of the subgroups,
providing the digital representation to an apparatus according to claim 10 to determine and provide a predicted technical application property of the polymer based on the digital representation,
comparing the predicted technical application property with the target technical application property and determining i) the synthesis specification as the optimal synthesis specification if the predicted technical application property lies within a predetermined range around the target application property, and ii) an amended synthesis specification taking the optimization constraints into account and repeating the optimization if the predicted technical application property lies outside a predetermined range around the target application property, and
a control signal generation unit for generating a control signal based on the optimal synthesis specification.

14. A computer program product for predicting a technical application property for a polymer, wherein the computer program product comprises program code means for causing the apparatus of claim 10 to execute the method according to claim 1.

15. A computer program product for training a machine learning based prediction model, wherein the computer program product comprises program code means for causing the apparatus of claim 13 to execute the method according to claim 9.

16. A system, wherein the system comprises i) a control signal comprising a synthesis specification of a polymer indicating one or more ingredients for producing the polymer, wherein the control signals are generated according to claim 5 and ii) the one or more ingredients indicated by the synthesis specification in the control signal.

17. A use of a control signal generated according to claim 5 for controlling a production process, in particular, a production process comprising the production of a polymer.

18. A control signal, wherein the control signal is generated according to claim 5.
